(19) United States
(12) Patent Application Publication
(10) Pub. No.: US 2020/0230293 A1
(43) Pub. Date: Jul. 23, 2020

(54) Tissue Engineered "Axon Fusion" for Immediate Recovery Following Axon Transection

(71) Applicant: THE TRUSTEES OF THE UNIVERSITY OF PENNSYLVANIA, Philadelphia, PA (US)

(72) Inventor: Daniel Kacy CULLEN, Media, PA (US)

(21) Appl. No.: 16/753,634

(22) PCT Filed: Oct. 5, 2018
(86) PCT No.: PCT/US18/54576

§ 371 Date: Apr. 3, 2020

Related U.S. Application Data
(60) Provisional application No. 62/569,255, filed on Oct. 6, 2017.

(51) Int. Cl. A61L 27/38; A61L 27/18; C12M 3/00; C12M 1/22; C12M 1/12; C12M 1/42

(52) U.S. Cl. CPC A61L 27/383; A61L 27/18; A61L 27/3878; A61L 27/3895; A61L 2430/32; C12M 23/10; C12M 25/02; C12M 35/04; C12M 21/08

(57) ABSTRACT

The present invention includes a composition comprising a tissue engineered axonal tract, as well as a method of making it. The invention also includes methods for treating nerve injury in a subject by contacting the site of nerve injury with a tissue engineered axonal tract, wherein the axons from the tissue engineered axonal tract fuse with axons from the subject.

### CROSS REFERENCE TO RELATED APPLICATIONS

The present application claims priority under 35 U.S.C. § 119(e) to U.S. Provisional Patent Application No. 62/569,255, filed Oct. 6, 2017, which is incorporated herein by reference in its entirety.

### STATEMENT REGARDING FEDERALLY SPONSORED RESEARCH OR DEVELOPMENT

This invention was made with government support under grant number W81XWH-16-1-0796 & W81XWH-15-1-0466 awarded by the U.S. Department of Defense. The government has certain rights in the invention.

### BACKGROUND OF THE INVENTION

Peripheral Nerve Injury (PNI) occurs with surprising frequency. It is reported in up to 3% of all trauma patients, increasing to 5% if plexus and root avulsion cases are included. PNI can also occur as a consequence of surgery, most often the transection of nerves during tumor resection. In total, the annual number of PNI procedures in the U.S. is estimated to be 558,862. In addition to civilian trauma, blast and penetrating injuries during wartime can result in major tissue and peripheral nerve loss, with craniomaxillofacial injuries accounting for more than 25% of wounded warriors treated in U.S. military facilities and extremity trauma accounting for as much as 79% of trauma cases. For patients requiring surgical reconstruction of an injured nerve, only 50% will achieve good to normal restoration of function, regardless of the repair strategy. Moreover, full functional recovery is considered impossible for major PNI—those injuries in which a large segment of a nerve is lost (i.e., >5 cm) or more proximal injuries (e.g., brachial plexus) that require extremely long distances for axonal regeneration to distal targets (e.g., hand). As such, patients who experience major PNI face the high probability of a residual functional deficit, even following state-of-the-art surgical reconstruction. For some, the deficit will be significant, debilitating and life altering (e.g., flail/nonfunctional arm or hand; decreased mobility; facial palsies impacting speech, eating). Tragically, if motor and sensory functions are not restored, the salvaged limb remains nonfunctional, insensate, often painful, and may ultimately necessitate amputation.

The inadequacy of current PNI repair options stems from the failure to overcome the long regenerative distances and times necessary to reinnervate distal targets. When a nerve is cut or deformed (i.e. stretched or crushed) such that axonal integrity is compromised, the axonal segment distal to the injury site rapidly degenerates—a phenomenon believed to be an inevitable consequence of being disconnected from its cell body. To restore function, axon regeneration must not only occur across the gap between the proximal and distal nerve stumps, but also through the entire distal nerve segment to end targets. Thus, PNI repair is ultimately a race against time, where long regenerative distances and slow axonal rates of regeneration (˜1 mm/day) create prolonged periods of denervation that negatively impact the capacity for axon regeneration as well as the ability of distal nerve structures to support regeneration (FIG. 1). Prolonged periods of denervation also negatively impact end targets and will ultimately and irrevocably render muscle unresponsive to reinnervation.

Current clinical practices center on “stealing” from healthy nerves to (partially) repair damaged nerves. Today, leading strategies for peripheral nerve reconstruction embrace a “robbing Peter to pay Paul” philosophy in which a healthy nerve is sacrificed to repair an injured nerve. For instance, the gold standard to bridge a nerve gap is the sensory nerve autograft. For over 30 years the field has tried to develop a bridging solution to replace the autograft and thus eliminate the need to harvest healthy donor nerve. To date, results have been disappointing as only markedly inferior products have been introduced [e.g., nerve guidance tubes (NGTs) such as Baxter's GEM NeuroTube® and Stryker's Neuroflex™; Axogen's acellular nerve allograft (ANA) Avance®]. As such, clinical use of these products is limited to noncritical sensory nerve injuries close to target (e.g., PNI in the hand or wrist), with virtually all motor and critical sensory nerves repaired using an autograft. To “babysit” the distal pathway (also referred to as “pathway protection”), the field has witnessed the advent of end-to-side coaptation procedures to temporarily innervate distal nerve structures and muscle. Despite promise in preclinical models, clinical implementation of end-to-side transfers has been selective. These procedures not only require an expendable donor nerve in close proximity to distal segments of injured nerve (which may not be available), but also typically result in a donor deficit. Often, these procedures are only used as a last resort in cases where the patient has not shown functional recovery many months following PNI repair. As such, these procedures are inherently compromised and have limited potential for regeneration and reinnervation due to the negative impact of chronic denervation on the distal pathway and end targets.

TENGs are living three-dimensional nerve constructs that consist of neurons and longitudinally aligned axonal tracts spanning discrete neuronal populations—thus mimicking aspects of the structure of the lost nerve. The ability to generate TENGs is based upon seminal discoveries regarding axon growth via continuous mechanical tension, also referred to as “stretch growth” (Smith et al., Tissue Eng 7, 131-139 (2001)). Stretch growth is a mechanism that can extend integrated axons (i.e. post-synaptic) at rapid rates using custom mechanobioreactors through controlled separation of two neuron populations. During stretch growth, individual axons gradually coalesce to form large axonal tracts, called fascicles, taking on a highly organized parallel orientation. This process can rapidly generate axon tracts of unprecedented lengths of 5-10 cm in 14-21 days, with no theoretical limit as to the final length (Pfister et al., J Neurosci 24, 7978-7983 (2004); Smith, Prog Neurobiol 89, 231-239 (2009), U.S. Pat. No. 6,365,15) (FIGS. 2A-2D). Three-dimensional TENGS are subsequently created by embedding the living axonal tracts in an extracellular matrix and removing from culture en masse (Pfister et al., J Neurosci Methods 153, 95-103 (2006); Huang et al., Tissue Eng Part A 15, 1677-1685 (2009)). It was demonstrated in rat and pig models of PNI, that allogeneic TENGs possess both bridging and babysitting properties and can overcome inherent limitations of nerve regeneration that cripple current repair options.

There remains a critical need to overcome the inherent limitations of nerve regeneration and chronic denervation in order to effectively treat nerve injuries. The present invention provides compositions and methods that satisfy these needs.

### SUMMARY OF THE INVENTION

In one aspect, the invention provides a method of treating nerve injury in a subject, the method comprising contacting a site of nerve injury with a tissue engineered axonal tract, wherein when the tissue engineered axonal tract contacts the site of nerve injury, at least one axon from the tissue engineered axonal tract fuses with at least one axon from the subject.

In another aspect, the invention provides a method of treating nerve injury in a subject, the method comprising severing a proximal stump at a site of nerve injury, contacting the proximal stump with a tissue engineered axonal tract, wherein when the tissue engineered axonal tract contacts the proximal stump, at least one axon from the tissue engineered axonal tract fuses with at least one axon from the proximal stump.

In another aspect, the invention provides a method of treating nerve injury in a subject, the method comprising contacting a site of nerve injury with a stretch-grown tissue engineered nerve graft (TENG), wherein when the TENG contacts the site of nerve injury, the at least one axon from the TENG fuses with at least one axon from the subject.

In another aspect, the invention provides a method of treating nerve injury in a subject, the method comprising contacting a site of nerve injury with a tissue engineered axonal tract and a stretch-grown tissue engineered nerve graft (TENG), wherein when the tissue engineered axonal tract contacts the site of nerve injury, at least one axon from the tissue engineered axonal tract fuses with at least one axon from the subject.

In various embodiments the method further comprises regeneration of a distal nerve segment in the subject.

In various embodiments, the nerve injury is selected from the group consisting of: peripheral nerve injury, brain injury, and spinal cord injury.

In various embodiments, the method further comprises the use of polyethylene glycol (PEG) to support nerve conduction and fusion of axons.

In various embodiments, the method further comprises the application of a hypotonic 50% by weight solution of PEG.

In various embodiments, the nerve injury is a result of a neurodegenerative disease.

In various embodiments, the TENG is a forced aggregation TENG.

In another aspect, the invention provides a composition comprising a tissue engineered axonal tract.

In various embodiments, the tissue engineered axonal tract is generated from a forced aggregation TENG.

In various embodiments, the invention provides a method of generating a tissue engineered axonal tract, the method comprising severing at least one neuronal cell body from a stretch-grown tissue engineered nerve graft (TENG), wherein when the at least one neuronal cell body is severed, a tissue engineered axonal tract is generated.

In various embodiments, the stretch-grown tissue engineered nerve graft (TENG) is a forced cell aggregation TENG.

In various embodiments, the invention provides a tissue engineered axonal tract made by the methods described herein.

### BRIEF DESCRIPTION OF THE DRAWINGS

The following detailed description of preferred embodiments of the invention will be better understood when read in conjunction with the appended drawings. For the purpose of illustrating the invention, there are shown in the drawings exemplary embodiments. It should be understood, however, that the invention is not limited to the precise arrangements and instrumentalities of the embodiments shown in the drawings.

FIG. 1 illustrates why peripheral nerve repair fails. Endogenous axon growth is often insufficient to functionally repair major PNI. Typical axon regeneration rates of 1 mm/day can require many months to years to reach distal end targets. Over this time, distal Schwann cells (SCs) lose their proregenerative capacity and eventually degenerate, ceasing axonal extension. In addition, target muscles undergo extreme atrophy and lose motor end plates, diminishing or even preventing re-innervation. Thus, slow axonal regeneration coupled with chronic distal denervation limit functional recovery.

FIGS. 2A-2D illustrate axon stretch-growth. FIGS. 2A-2C show a schematic of the stretch-growth process. FIG. 2A shows neurons cultured on 2 overlapping membranes, (FIGS. 2B-2C) which are gradually pulled apart in custom mechano-bioreactors to induce stretch-growth of the spanning axons. FIG. 2D shows fluorescently labeled axons stretch-grown to 5 cm over 2 weeks.

FIGS. 3A-3C illustrate immediate nerve conduction following fusion of tissue engineered axonal tracts. FIG. 3A shows nerve conduction from the proximal to the distal nerve measured immediately following polyethylene glycol (PEG)-mediated anastomosis of tissue-engineered axons with host axons. FIG. 3B shows positive controls. FIG. 3C shows negative controls. Re-opposition of proximal and distal stump (FIG. 3B) with PEG treatment yielded immediate nerve conduction whereas re-opposition (FIG. 3C) without PEG treatment failed to support nerve conduction.

FIGS. 4A-4B illustrate the finding that fusion of tissue engineered axonal tracts partially prevents host axon degeneration in vivo. Tissue-engineered axonal tracts were used to directly “splice-in” across a missing segment of sciatic nerve in rats, either with PEG or without (negative control), and nerve morphometry was assessed at 14 days post-transection. FIG. 4A shows in both groups, host axons were present in the segment proximal to the transection site. FIG. 4B shows spared host axons were observed distal to the repair site in animals treated with PEG, but not in animals without PEG, demonstrating host axonal fusion with tissue engineered axonal tracts.

FIGS. 5A-5F show evidence of structural fusion of excised sciatic nerve axons and tissue engineered axonal tracts. Following immediate opposition of excised sciatic nerve and tissue engineered axonal tracts, PEG-mediated fusion resulted in axonal continuity at 1 day post-fusion. This was demonstrated by axons containing both LY (transported through the excised host axons) and mCherry (expressed exclusively by the TENG neurons/axons), demonstrating fusion and continuity between sciatic and TENG axons.

FIG. 6 illustrates axonal fusion in tissue-engineered axonal tracts in vitro. 3-D Tissue Engineered Nerve Grafts (TENGs) were transected in vitro, treated with PEG, and immediately re-opposed. A fluorescent live-dead stain was then applied, which labels metabolically active axons in green. Confocal microscopy revealed that a subset of axons were continuous (white circles), whereas others showed disconnection (red circles). The continuous axons at 30-60 minutes post-transection suggests that at least a subset of the axons were able to re-fuse following PEG treatment.

FIG. 7 illustrates the generation of tissue engineered axonal tracts for direct fusion with disconnected host axons.

FIG. 8 illustrates one non-limiting application of the present invention in which functional axons are “spliced in” across severed nerve regions.

FIG. 9 illustrates another non-limiting application of the present invention comprising complete replacement of the distal nerve segment all the way to the target.

FIG. 10 illustrates yet another non-limiting application of the present invention comprising dual “splicing” and conventional bridging across severed nerve regions.

FIG. 11 depicts a cartoon showing the cell “forced aggregation method” as applied to neuron culture, axon stretch-growth, and TENG construction. Dissociated motor neurons are harvested from embryonic rat spinal cords and plated in pyramid shaped wells, centrifuged, and incubated in plating media overnight to allow aggregates of motor neurons to form. After 24 hours, individual aggregates are taken from the wells and placed on either side of the interface of a thin and a thick membrane. After 5-7 DIV, once the two populations of aggregates have formed axonal connections, one of the populations is pulled back in micron-sized increments using a stepper motor to allow long aligned motor axons to form. Once the desired length is reached, the axonal construct is encapsulated in a proteinaceous matrix and inserted into a nerve guidance tube for transplantation into a peripheral nerve injury model.

FIGS. 12A-12C are micrographs of the formation and tissue culture of neuronal “forced aggregates”. FIG. 12A shows an aggregate method to create neuron “spheres” with controlled size (and hence neuronal density). FIG. 12B shows motor neuron aggregates at 3 days post-plating. FIG. 12C shows motor neuron aggregate and axonal outgrowth.

FIG. 13 shows phase contrast micrographs of the culture architecture for motor neurons cultured following dissociation (old method) versus “forced aggregates” (new method). Depicted are motor neurons in culture at one day post-plating.

FIGS. 14A-14C depict motor neuron-specific markers P75 and choline acetyl transferase (ChAT) in cultured motor neurons. FIG. 14A depicts dissociated motor neurons labeled for the nuclear counterstain HOECHST (blue), the neuronal marker beta-tubulin III (green), and motor neuron marker P75 (red), also seen in the overlay image. Scale: 250 μm. FIG. 14B depicts aggregated motor neurons labeled with HOECHST nuclear counterstain (blue) and express the motor neuron marker P75 (red), which is also seen in the overlay. Scale: 500 μm. FIG. 14C depicts aggregated motor neurons labeled for nuclear counterstain (blue), beta-tubulin III (green), and another motor neuron marker ChAT (red), seen in the overlay image. Scale: 500 μm.

FIGS. 15A and 15B depict representative confocal micrographs demonstrating significantly increased axonal outgrowth when “forced aggregate” motor neurons are in co-culture with sensory neuron ganglia (FIG. 15B), versus dissociated motor neurons in co-culture with sensory neuron ganglia (FIG. 15A).

FIGS. 16A-16C depict a demonstration of motor neuron stretch growth. Motor neurons were harvested from embryonic rat spinal cords and dissociated using bovine serum albumin (BSA) density gradients to acquire a relatively pure population of motor neurons. FIG. 16A depicts phase contrast images of successful axon stretch grown of dissociated motor neurons following application of mechanical tension at relatively slow rates of 0.1-0.3 mm/day; scale: 1000 μm. FIGS. 16B and 16C each depict a higher magnification of stretch grown axons from motor neurons showing alignment and fasciculation.

FIGS. 17A-17F are a series of images illustrating that dissociated motor axons are unable to tolerate higher rates of mechanical stress. FIG. 17A depicts dissociated motor neurons plated within a mechanobioreactor, and axons were subject to application of mechanical tension at a rate of 1 mm/day, FIGS. 17B-17E show this leading to snapping of the axons. FIG. 17F shows a comparison, DRG axons are routinely subjected to rates of mechanical stress ranging from 1-3 mm/day, resulting in long, stretch grown axon tracts.

FIGS. 18A-18C are phase contrast micrographs demonstrating axonal “stretch-growth” from motor neuron “forced aggregates” in co-culture with sensory neuron ganglia. This methodology is crucial to developing mixed motor-sensory TENGs in order for axonal fusion and immediate functional recovery in injured mixed motor-sensory nerves. FIG. 18A is a zoomed out image and FIGS. 18B and 18C are higher magnifications of the insets.

FIGS. 19A-19J is a series of images depicting a motor neuron aggregation method allows for higher rates of axonal stretch-growth. Rat motor neurons were forced into neuronal aggregates and plated in custom-built mechanobioreactors. FIG. 19A is a phase contrast image of the stretch-grown motor axons after tension was applied at a rate of 1 mm/day for 1 day, and axons have stretched to approximately 1 mm. FIG. 19B shows that motor axons were able to be stretch-grown at a rate of 1 mm/day over at least several days, resulting in fascicularized bundles of motor axons spanning the length of 1 cm. Scale A and B: 1000 μm. FIGS. 19C-19F depict a confocal reconstruction of motor neuron aggregate and FIGS. 19G-19J depict axonal sections, showing nuclear stains (HOECHST; FIG. 19C, 19G), axons (beta-tubulin; FIG. 19D, 19H), motor neuron specific marker (p′75; FIG. 19E, 19I), and all channels merged (FIG. 19F, 19J). Scale: 250 μm. The higher rates of axonal stretch-growth enabled by the forced aggregate method allows for the generation of motor neuron-based tissue engineered nerve grafts (TENGs) capable of bridging critical length nerve deficit in one-tenth the time as stretch-growth of axons from dissociated motor neurons.

FIGS. 20A and 20B depict a mixture of aggregates from motor neurons and sensory neurons exhibited robust axon stretch growth and fasciculation out to at least 1 cm. FIG. 20A depicts alternating, differentially labeled sensory dorsal root ganglia (DRG; red) and spinal motor neuron aggregates of similar size that were plated prior to application of displacement. The two neuronal populations were differentially labeled during the aggregation process using AAV tagged GFP to label motor neurons and AAV tagged mCherry to label sensory neurons. FIG. 20B depicts how displacement rates of 1 mm/day yield long sensory and motor axons spanning 1 cm. Scale: 1000 μm.

FIGS. 21A-21D depict transplantation of a sensory neuron/axon TENG into 1 cm sciatic nerve injury model in rats. FIGS. 21A-D are confocal reconstructions of nerve regeneration across TENGs. FIG. 21A depicts a full longitudinal section labeled for host axons (neurofilament, purple), host Schwann cells (S100, red), and TENG neurons/axons (GFP, green). Robust host axon axonal infiltration was onserved from the proximal stump. FIGS. 21B-21D are each a higher magnification of regions of interest from A. FIGS. 21B and 21C depict axonal projections into the distal stump from TENG neurons interact with host Schwann cells. FIGS. 21C and 21D depict robust TENG neuron and axon survival to facilitate the regenerative process as well as host Schwann cell infiltration from the distal stump. Scale: 100 μm.

FIGS. 22A-22E depict transplantation of mixed motor neuron—sensory neuron TENG into 1 cm sciatic nerve injury model in rats. Each of FIGS. 22A-E show confocal reconstructions of nerve regeneration across TENGs. FIG. 22A depicts a full longitudinal section labeled for host axons (neurofilament, purple), host Schwann cells (S100, red), and TENG neurons/axons (GFP, green). FIGS. 22B-22E are each a higher magnification of regions of interest from A. FIG. 22B depicts a host axon neurofilament showing robust axonal infiltration. FIGS. 22C and 22D depict robust TENG neuron and axon survival to facilitate the regenerative process. FIG. 22E depicts robust host Schwann cell infiltration from the distal stump. Scale: 100 μm.

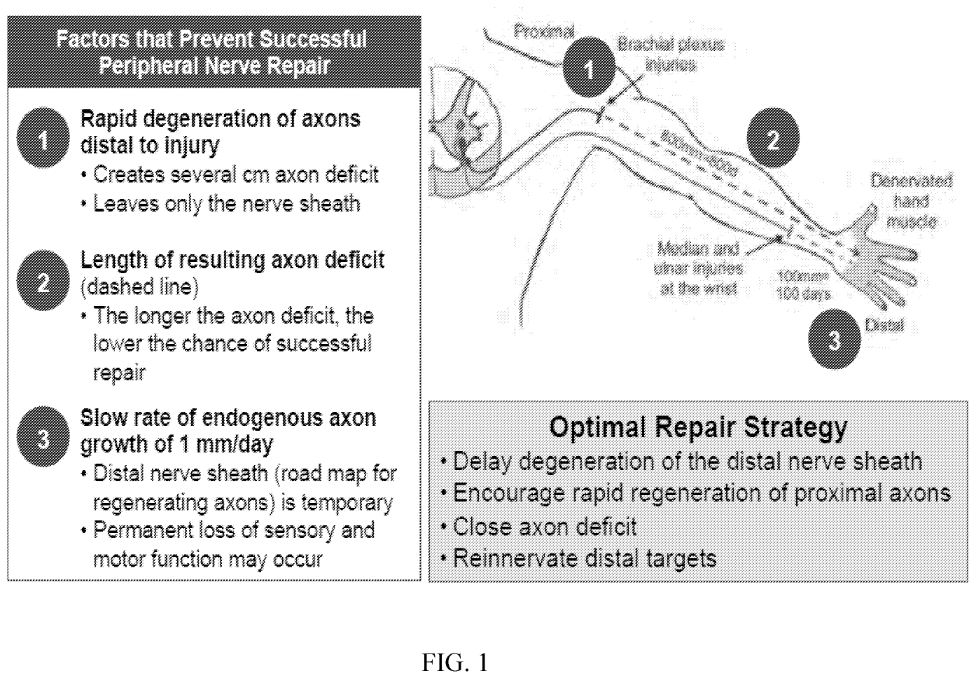

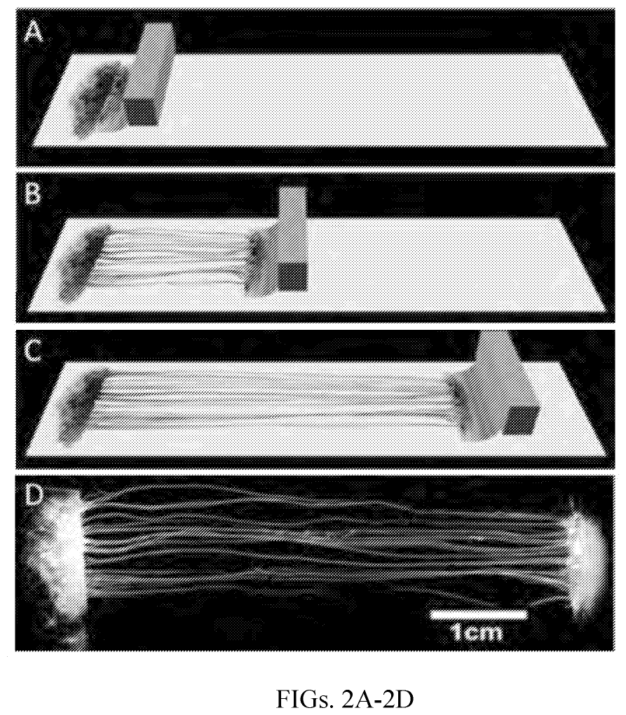

### DETAILED DESCRIPTION OF THE INVENTION

### Definitions

Unless defined otherwise, all technical and scientific terms used herein have the same meaning as commonly understood by one of ordinary skill in the art to which the invention pertains. Although any methods and materials similar or equivalent to those described herein can be used in the practice for testing of the present invention, the preferred materials and methods are described herein. In describing and claiming the present invention, the following terminology will be used.

It is also to be understood that the terminology used herein is for the purpose of describing particular embodiments only, and is not intended to be limiting.

As used herein, the singular form “a,” “an,” and “the” include plural references unless the context clearly dictates otherwise.

“About” as used herein when referring to a measurable value such as an amount, a temporal duration, and the like, is meant to encompass variations of ±20% or ±10%, more preferably ±5%, even more preferably ±1%, and still more preferably ±0.1% from the specified value, as such variations are appropriate to perform the disclosed methods. Unless otherwise clear from context, all numerical values provided herein are modified by the term about.

As used in the specification and claims, the terms “comprises,” “comprising,” “containing,” “having,” and the like can have the meaning ascribed to them in U.S. patent law and can mean “includes,” “including,” and the like.

As used herein, the term “cylinder” or “cylindrical” includes a surface consisting of each of the straight lines that are parallel to a given straight line and pass through a given curve. In some embodiments, cylinders have an annular profile. In other embodiments, the cylinder has a cross-section selected from the group consisting of: a square, a rectangle, a triangle, an oval, a polygon, a parallelogram, a rhombus, an annulus, a crescent, a semicircle, an ellipse, a super ellipse, a deltoid, and the like. In other embodiments, the cylinder is the starting point of a more complex three-dimensional structure that can include, for example, complex involutions, spirals, branching patterns, multiple tubular conduits, and any number of geometries that can be implemented in computer-aided design, 3-D printing, and/or in directed evolutionary approaches of secretory organisms (e.g., coral), including of various fractal orders.

A “disease” is a state of health of an animal wherein the animal cannot maintain homeostasis, and wherein if the disease is not ameliorated then the animal's health continues to deteriorate. In contrast, a “disorder” in an animal is a state of health in which the animal is able to maintain homeostasis, but in which the animal's state of health is less favorable than it would be in the absence of the disorder. Left untreated, a disorder does not necessarily cause a further decrease in the animal's state of health.

“Effective amount” or “therapeutically effective amount” are used interchangeably herein, and refer to an amount of a compound, formulation, material, or composition, as described herein effective to achieve a particular biological result or provides a therapeutic or prophylactic benefit.

“Forced aggregation” and “forced cell aggregation” are used interchangeably herein, and refer to a method of forming “aggregates” or “spheres” of neurons by centrifugation in inverted pyramidal micro-wells.

“Forced aggregation TENG” and “forced cell aggregation TENG” are likewise used interchangeably to refer to a TENG that is stretch grown from an aggregate or sphere of neurons formed by forced aggregation.

By the term “modified” as used herein, is meant a changed state or structure of a molecule or cell of the invention. Molecules may be modified in many ways, including chemically, structurally, and functionally. Cells may be modified through the introduction of nucleic acids.

As used herein, “neurological disorder” refers to any disorder of the body's nervous system, which includes the nerves, brain and spinal cord. In certain embodiments, neurological disorders can arise from injury to the nerves, brain, or spinal cord.

As used herein, “neurodegenerative condition” refers to a condition in which there is a loss of structure or function of neurons, including death of neurons. Examples of neurodegenerative conditions include but are not limited to: Alzheimer's disease, Parkinson's disease, Huntington's disease, prion disease, motor neurone diseases, spinocerebellar ataxia, spinal muscular atrophy, amyotrophic lateral sclerosis (ALS), encephalitis, epilepsy, head and brain malformations, and hydrocephalus.

Unless specifically stated or obvious from context, the term “or,” as used herein, is understood to be inclusive.

“Parenteral” administration of an immunogenic composition includes, e.g., subcutaneous (s.c.), intravenous (i.v.), intramuscular (i.m.), or intrasternal injection, or infusion techniques.

A “subject” or “patient,” as used therein, may be a human or non-human mammal. Non-human mammals include, for example, livestock and pets, such as ovine, bovine, porcine, canine, feline and murine mammals. Preferably, the subject is human.

The term “therapeutic” as used herein means a treatment and/or prophylaxis. A therapeutic effect is obtained by suppression, remission, or eradication of a disease state.

“Tissue engineered axonal tracts” refer to living axonal tracts generated from TENGs, in which the neuronal cell bodies have been severed leaving only axonal tracts. In various embodiments the TENG may have been generated from any sub-type of neuron, including but not limited to neurons from the peripheral nervous system (e.g., spinal motor, sensory dorsal root ganglia), central nervous system (e.g., glutamatergic, GABAergic, dopaminergic, serotonergic), and autonomic nervous system (e.g, ganglionic norepinephrinergic, acetycholinergic, or dopaminergic).

“Tissue-Engineered Nerve Grafts (TENGs)” is used interchangeably herein with the term “stretch-grown TENG” and refers to living three-dimensional nerve constructs that consist of neurons, including neuronal cell bodies, and longitudinally aligned axonal tracts.

To “treat” a disease as the term is used herein, means to reduce the frequency or severity of at least one sign or symptom of a disease or disorder experienced by a subject.

Ranges: throughout this disclosure, various aspects of the invention can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

### DESCRIPTION

The present invention includes compositions and methods for treating nerve injury in a subject. In one embodiment, the invention includes a tissue engineered axonal tract. In another embodiment, the invention discloses a method for generating a tissue engineered axonal tract. In other embodiments, the invention includes methods for treating nerve injury in a subject by contacting the site of nerve injury with a tissue engineered axonal tract, wherein when the tissue engineered axonal tract contacts the site of nerve injury, at least one axon from the tissue engineered axonal tract fuses with at least one axon from the subject.

### Tissue Engineered Axonal Tracts

The present invention includes methods of making and using tissue engineered axonal tracts. In one aspect, the invention includes a composition comprising a tissue engineered axonal tract. Other aspects include methods of generating tissue engineered axonal tracts and other aspects include using issue engineered axonal tracts to treat nerve injry.

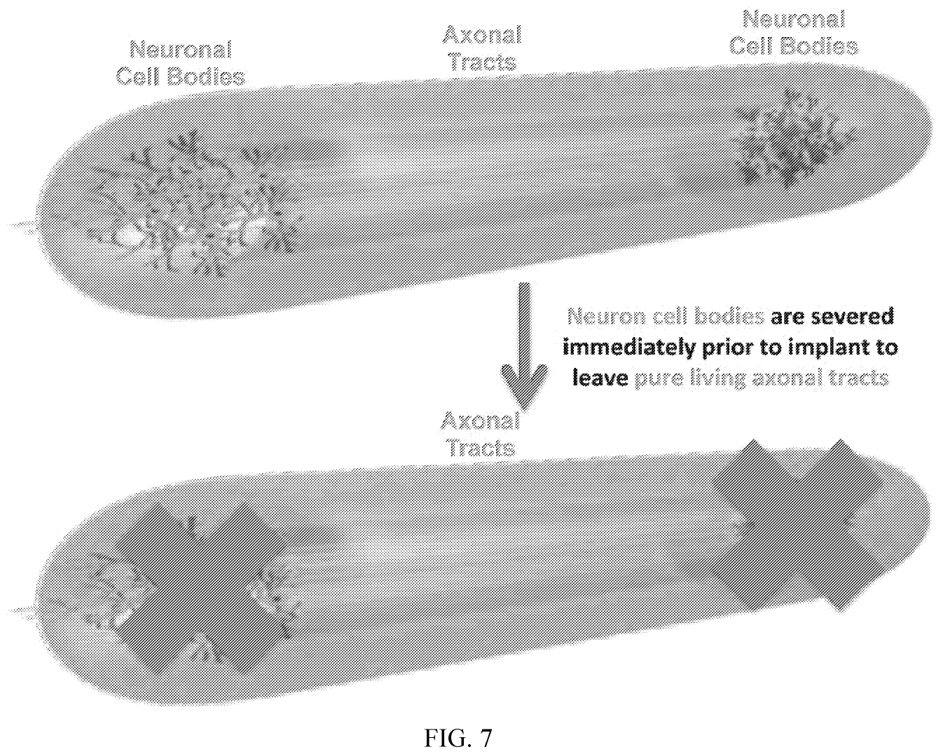

In generating a tissue engineered axonal tract, the starting biomass can be a tissue engineered construct consisting of 3-D neurons and axonal tracts. An example of such starting biomass includes, but is not limited to, Tissue-Engineered Nerve Grafts (TENGs). The neurons in the starting biomass include neuronal cell bodies. These neuronal cell bodies are subsequently severed, leaving pure living axonal tracts or “tissue engineered axonal tracts” (FIG. 7).

Tissue engineered axonal tracts can be made by the methods disclosed herein. In one embodiment, tissue engineered axonal tracts are made by the method comprising severing at least one neuronal cell body from a stretch-grown tissue engineered nerve graft (TENG), wherein when the at least one neuronal cell body is severed, a tissue engineered axonal tract is generated. In another embodiment, a tissue engineered axonal tract is generated from a stretch-grown tissue engineered nerve graft (TENG) generated via forced cell aggregation.

### Generating TENGs and Tissue Engineered Axonal Tracts Via “Forced Cell Aggregation”

The ability to generate TENGs and tissue engineered axonal tracts consisting of motor and/or sensory axons with robust axonal outgrowth and hardiness for axonal “stretch-growth” is necessary for the immediate axonal fusion and repair of injured motor and sensory axons. Described herein is the demonstration of stretch growth of robust motor axon fascicles following the creation of dense motor neuron spheres using a method referred to as “forced aggregation”.

It has been observed that robust axonal “stretch-growth” can be achieved using axons extending from embryonic sensory neurons in dorsal root ganglia, at least partially due to thick axonal fascicles projecting from dense clusters of neurons naturally found in ganglia. However, neurons from adult ganglia or the adult or embryonic central nervous system (i.e. brain and spinal cord) require enzymatic dissociation for survival in culture. Dissociated neurons generally extend finer neurites/axons (see FIGS. 13-14) than neurons in ganglia. Moreover, these finer axons projecting from dissociated neurons generally are not as robust following stretch-growth, resulting in a lower yield of stretch-grown axons, even when slower rates are used to prevent breakage (thus TENG construction takes longer to achieve desirable lengths, is therefore more costly, yet still has a reduced axonal yield).

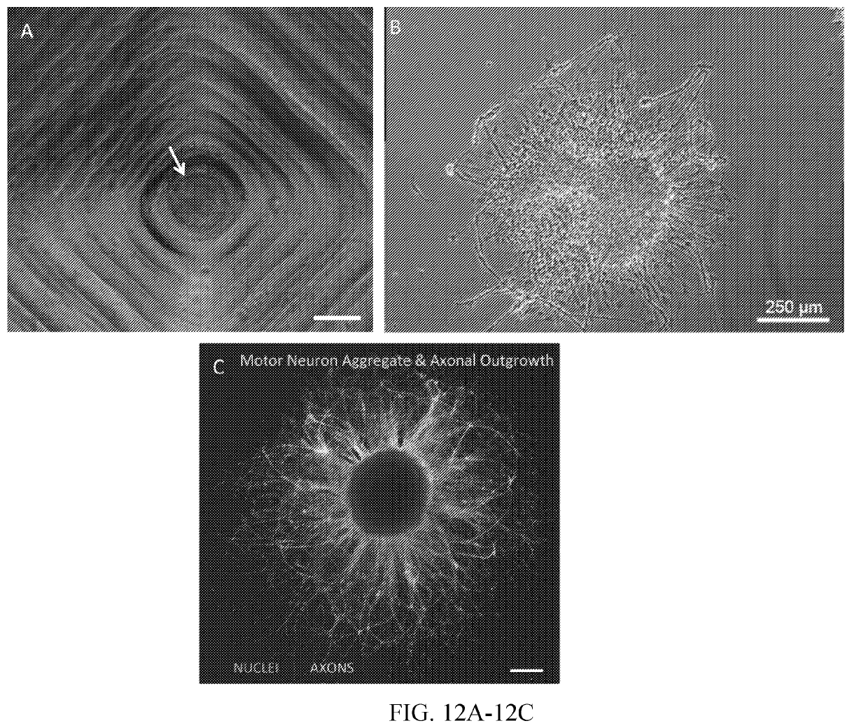

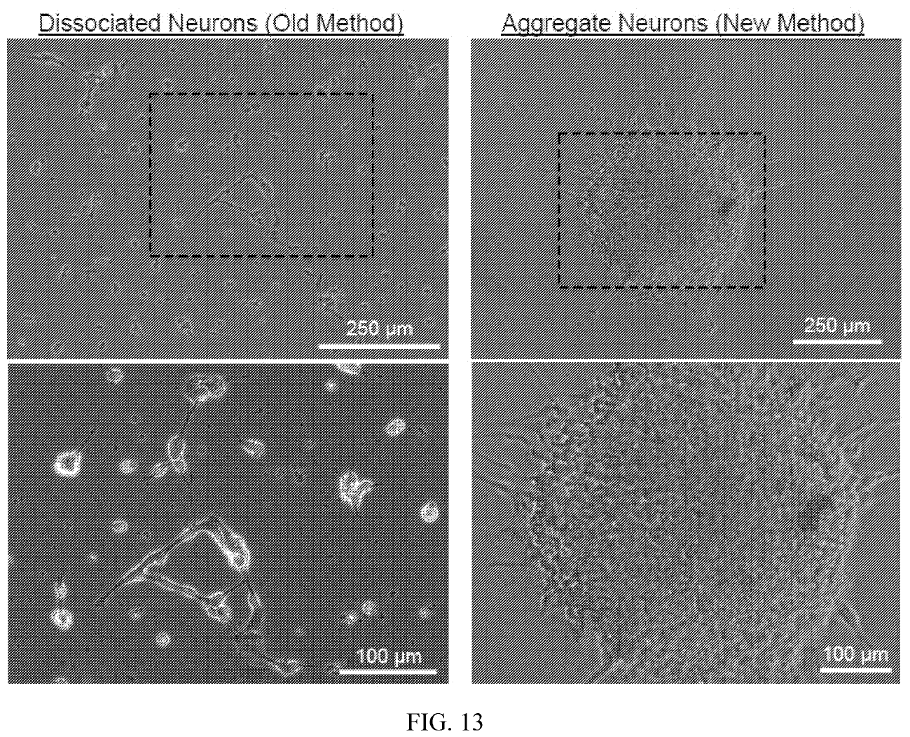

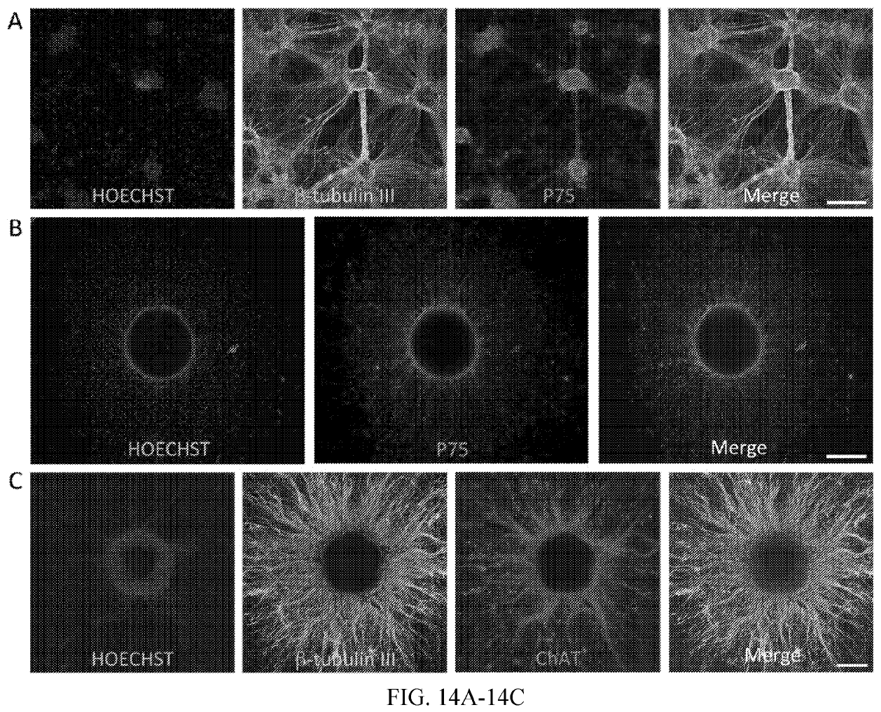

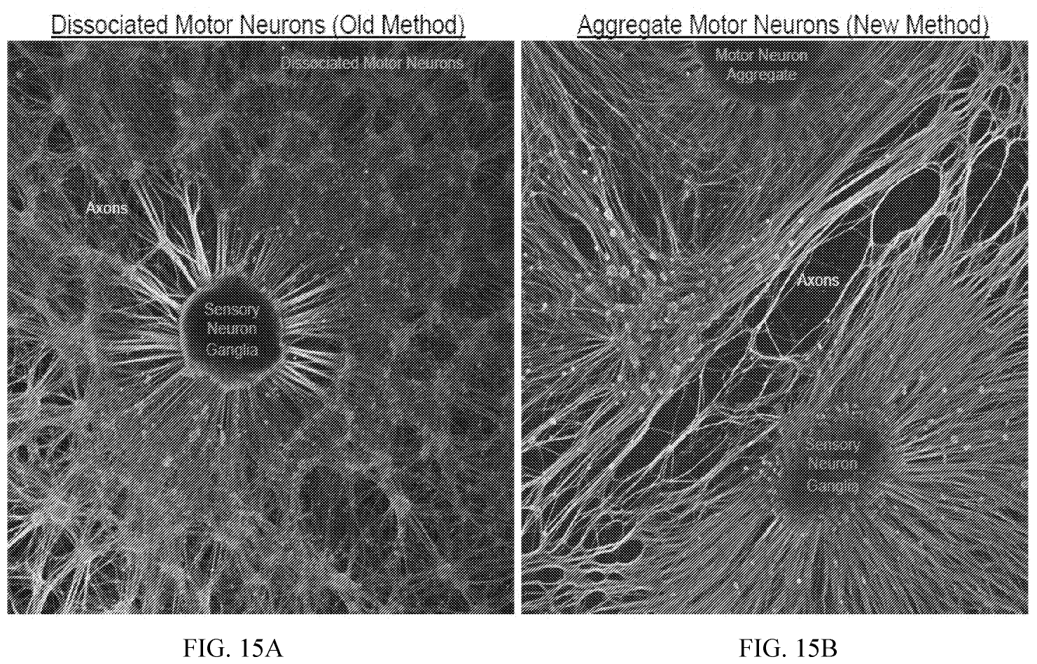

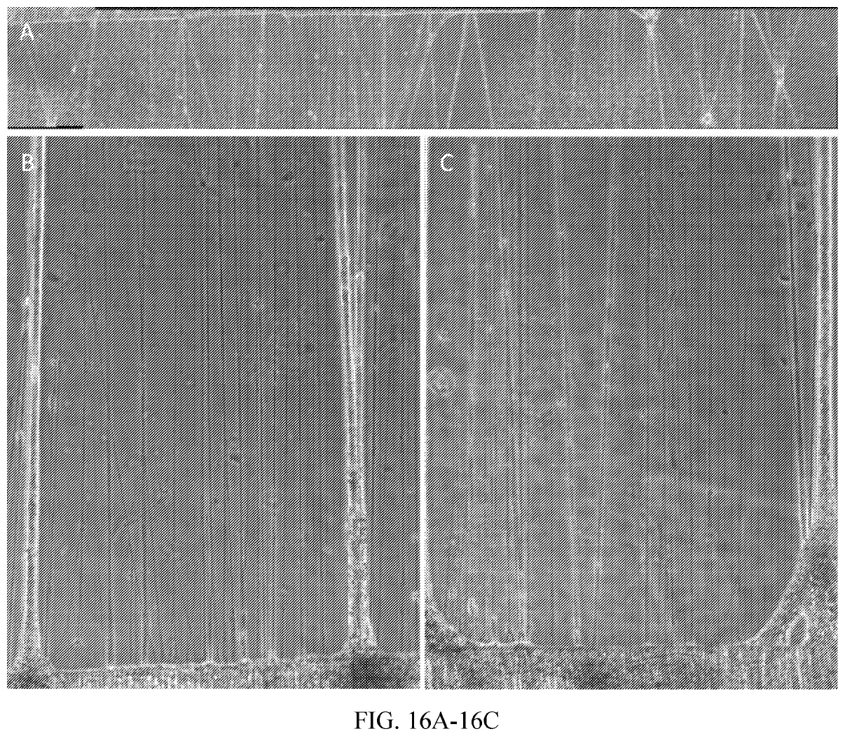

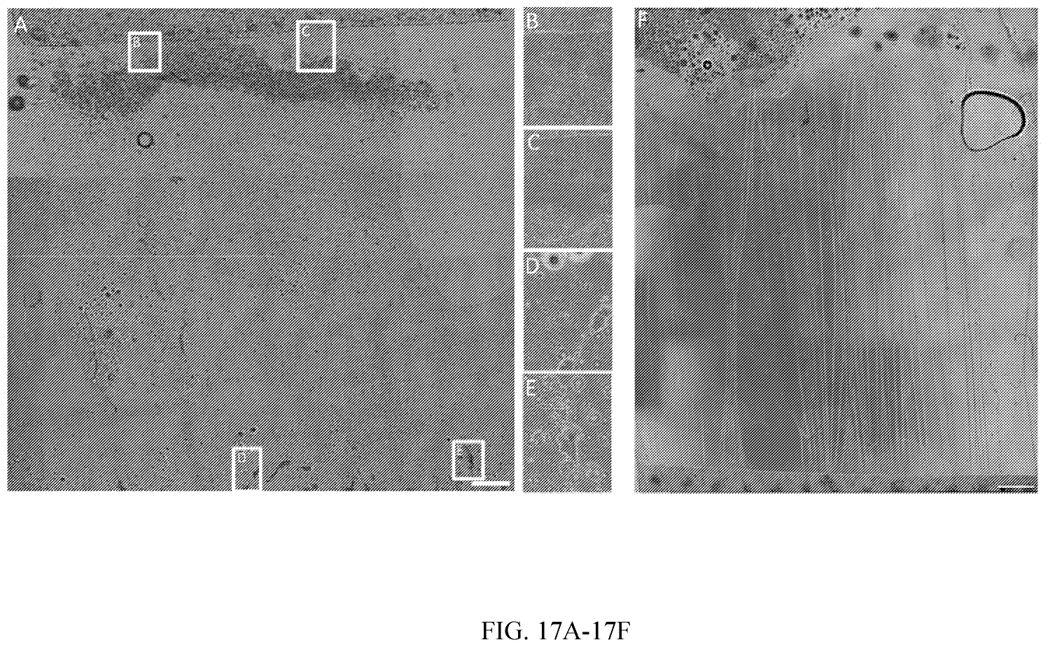

To address this issue, a novel tissue engineering methodology was devised and validated to more consistently create robust axonal outgrowth, axonal “stretch-growth”, and ultimately TENG formation from neuronal populations that require dissociation. As described herein, “forced cell aggregation” was utilized within custom-build pyramidal micro-wells to create “aggregates” or “spheres” of neurons with precise control of the number of neurons—and hence diameter—per aggregate/sphere (FIGS. 12A-12C). By way of non-limiting example, dissociated neurons were transferred to a chamber containing an array of inverted pyramid micro-wells made in PDMS (Sylguard 184, Dow Corning) cast from a custom-designed 3D printed mold. The wells were then centrifuged at 200 g for 5 min. This centrifugation resulted in forced aggregation of motor neurons (or any other cell type if desired) with precise control of the number of neurons per aggregate/sphere based on the density of neurons used and the volume added to each well. In one non-limiting example, 12μL of neuronal suspension per well at a density of 300,000-400,000 cells/mL was suitable to create aggregate/spheres of appropriate diameter for seeding, neuronal survival, axonal extension, and axonal “stretch-growth”. In another embodiment, the neuronal suspension can be at a density of 10,000-1,000,000 cells/mL. In yet another embodiment, the neuronal suspension can be at a density of 100,000-500,000 cells/mL.

The aggregates/spheres were carefully placed at the towing membrane—base membrane border in custom mechanobioreactors and allowed to adhere at 37° C., 5% CO₂, before the chambers were flooded with media. The media for motor neuron culture and axon stretch-growth was Neurobasal media with 10% FBS+hydrocortisone, growth factor cocktail (BDNF, CNTF, Cardiotrophin 1, GDNF), B-27, L-glutamine, Sodium Pyruvate, forskolin, mitotic inhibitors, isobutylmethylxanthine, and beta mercaptoethanol; this media was “glial conditioned” prior to use in motor neuron cultures. For motor neurons in co-culture with dorsal root ganglia (sensory) (DRG) neurons, the above media was further supplemented with NGF and glucose. Seeded mechanobioreactors were allowed to grow over at least several days in vitro prior to engaging the micro-stepper motor for axonal-stretch growth. Certain methods described herein are based on their application with spinal motor neurons; however, similar methods are applicable for “forced aggregation” and culture of any other neuronal subtype requiring dissociation.

### Neuronal Subtypes

The neurons useful for the compositions and methods provided herein include all neuronal subtypes, including but not limited to PNS motor or sensory, CNS, and stem cells (e.g., induced pluripotent stem cells, embryonic stem cells, and the like) differentiated into a neuronal phenotype. In one embodiment of the present invention, neurons are derived from any cell that is a neuronal cell (e.g., cortical neurons, dorsal root ganglion neurons or sympathetic ganglion neurons) or is capable of differentiating into a neuronal cell (e.g., stem cell). The neurons may be autologous, allogenic, or xenogenic with reference to the subject.

In certain embodiments, the neurons are peripheral or spinal cord neurons including dorsal root ganglion neurons or motor neurons. In certain embodiments, the neurons are from brain, including but not limited to, neurons from the cerebral cortex, thalamus, hippocampus, striatum, substantia nigra and cerebellum. In certain embodiments, primary cerebral cortical neurons include but are not limited to neurons from layers II, III, IV, V, and/or VI of the cortex (separately or in any combination thereof), neurons from the visual cortex, neurons from the motor cortex, neurons from the sensory cortex, and neurons from the entorhinal cortex.

Neurons useful in the invention may be derived from cell lines or other mammalian sources, such as donors or volunteers. In one embodiment, the neurons are human neurons. In one embodiment, the neurons are non-human mammalian neurons, including neurons obtained from a mouse, rat, dog, cat, pig, sheep, horse, or non-human primate. In one embodiment, the neurons are cortical neurons, hippocampal, neurons, dorsal root ganglion neurons or sympathetic ganglion neurons. In another embodiment, neurons are derived from immortalized cell lines that are induced to become neuron-like (e.g., NT2, PC12). In one embodiment, the neurons are neurons derived from a cadaver. In another embodiment, the neurons are neurons derived from patients who have undergone ganglionectomies, olfactory epithelium biopsy, temporal lobectomy, tumor margin resection, peripheral nerve biopsy, brain biopsy, ventricular shunt implantation with biopsy, or other clinical procedure. Furthermore, the neurons may be singular, integrated neurons or a plurality of integrated neurons (i.e., an integrated nerve bundle).

In certain embodiments, the neurons are cultured in vitro or ex vivo. Culture of the neurons can be performed under suitable conditions to promote the growth of axons. Those conditions include, without limitation, the appropriate temperature and/or pressure, electrical and/or mechanical activity, force, the appropriate amounts of O₂ and/or CO₂, an appropriate amount of humidity, and sterile or near-sterile conditions. For example, the cells may require a nutritional supplement (e.g., nutrients and/or a carbon source such as glucose), exogenous hormones or growth factors, and/or a particular pH. Exemplary cell culture media that can support the growth and survival of the neuron includes, but is not limited to, NEUROBASAL® media, NEUROBASAL® A media, Dulbecco's Modified Eagle Medium (DMEM), and Minimum Essential Medium (MEM). In certain embodiments, the culture medium is supplemented with B-27® supplements. In certain embodiments, the culture medium may contain fetal bovine serum or serum from another species at a concentration of at least 1% to about 30%, or about 5% to about 15%, or about 10%. In certain embodiments, the media is “glial conditioned”.

### Methods of Treating Nerve Injury

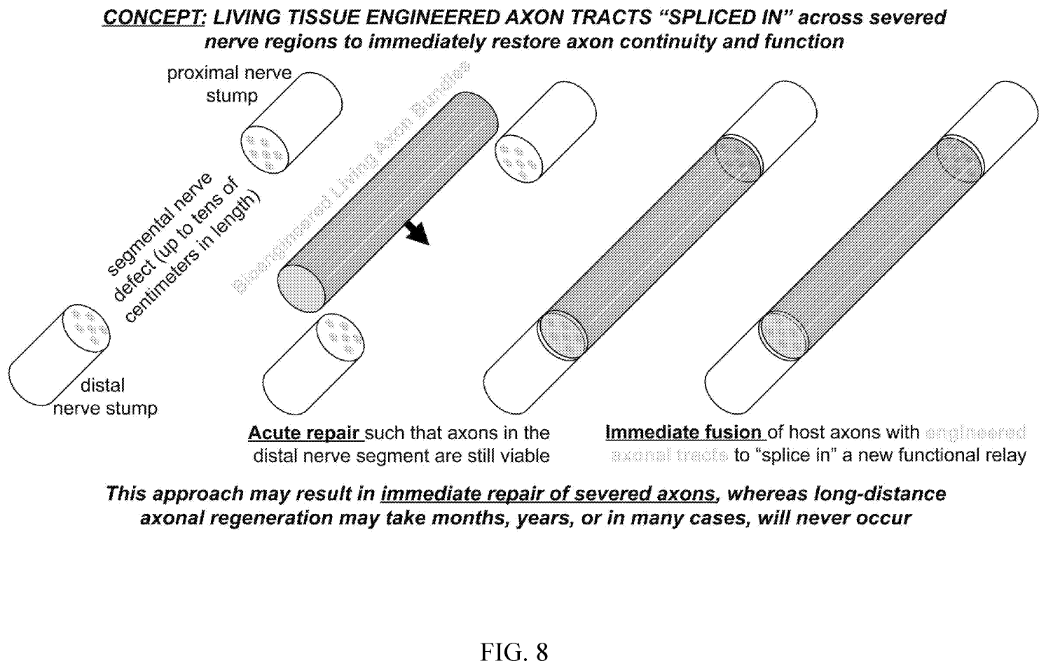

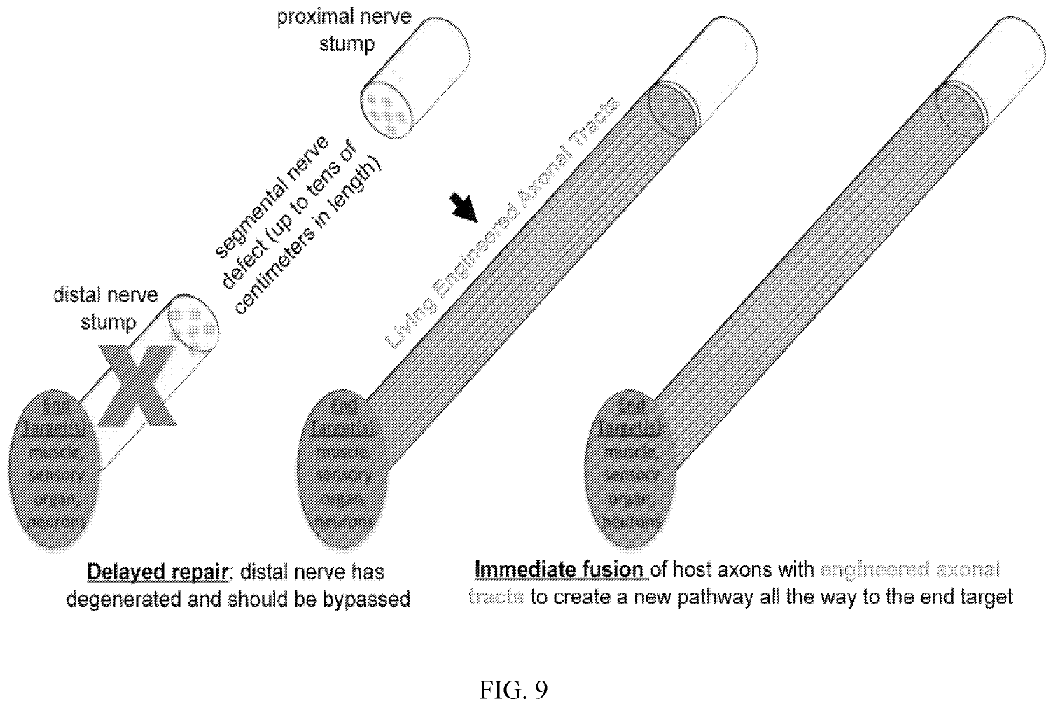

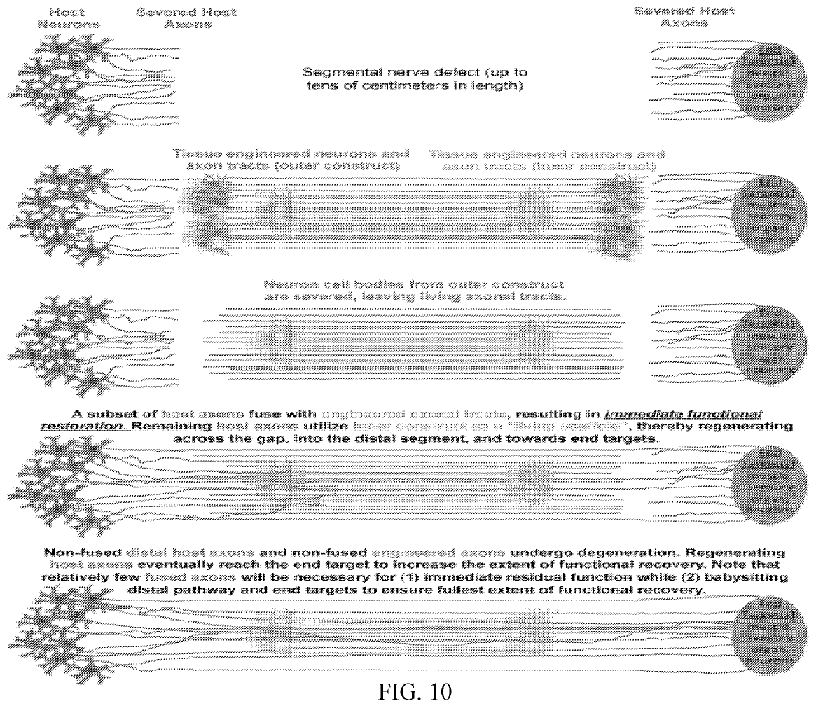

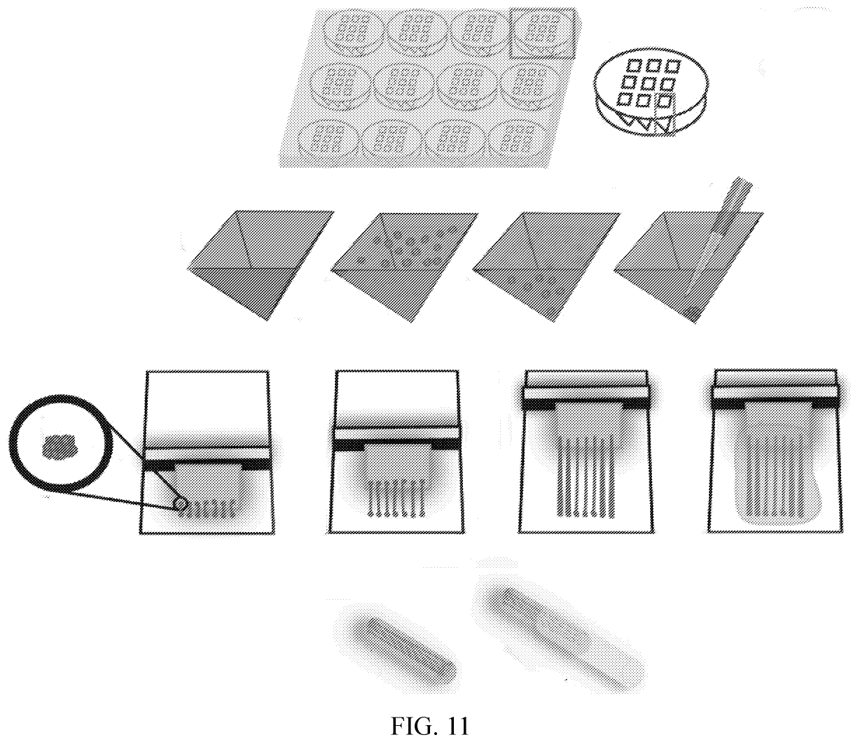

The present invention includes methods for treating nerve injury in a subject. In certain embodiments, the methods comprise contacting the site of nerve injury with a tissue engineered axonal tract. Tissue engineered axonal tracts can be neurosurgically implanted for axonal replacement. Non-limiting applications for tissue engineered axonal tracts, and tissue engineered axonal tracts in combination with TENGs include: Acute repair of peripheral nerve injury (PNI) (Example 4, FIG. 8 and FIG. 10), creation of replacement nerves or nerve segments for repair of chronic PNI (i.e. delayed repair) (Example 4, FIG. 9), creation of replacement nerves or nerve segments to treat chronic neuropathy (e.g., diabetic nerve damage, Charcot-Marie-Tooth disease), acute or delayed repair of spinal cord injury (SCI), treatment of motor neuron disease (e.g., ALS), traumatic brain injury, white matter stroke, brain tumor excision (e.g., glioblastoma), and neurodegenerative disease (e.g. ALS/motor neuron disease, Parkinson's, Alzheimer's).

Tissue engineered axonal tracts can be used to treat subjects with segmental nerve defects up to tens of centimeters in length between the proximal and distal nerve stumps. In one embodiment, tissue engineered axonal tracts are “spliced in” across the severed nerve region. In this case, axons in distal nerve segment are still viable and there is an immediate fusion of host axons with the axons from the tissue engineered axonal resulting in “splicing in” a new functional relay and acute repair of the nerve (FIG. 8).

In another application, the distal nerve stump is degenerated but can be bypassed by fusing host axons with tissue engineered axonal tracts to create a new pathway to the end target (i.e distal nerve) resulting in delayed repair of the nerve (FIG. 9).

In yet another application—there can be dual “splicing” and conventional bridging across the severed nerve region (FIG. 10). One embodiment involves two sets of TENGs: one set arranged as an inner construct and another set arranged as an outer construct. The neuron cell bodies from the outer construct are severed leaving only axonal tracts (tissue engineered axonal tracts). A subset of the host axons will fuse with the tissue engineered axonal tract, resulting in immediate functional restoration. The remaining host axons utilize the inner construct (TENG) as a “living scaffold”, thereby regenerating across the gap, into the distal segment, and towards the end target. Non-fused distal host axons and non-fused engineered axons undergo degeneration. Regenerating host axons eventually reach the end target to increase the extent of functional recovery. Relatively few fused axons will be necessary for immediate residual function while babysitting distal pathway and end targets to ensure the fullest extent of functional recovery.

Nerve injury treatment regimens utilizing tissue engineered axonal tracts and TENGs can vary and can be determined by one of ordinary skill in the art. In one embodiment, treatment occurs with TENGs comprised of tens of neurons, thousands of neurons, millions of neurons, tens of millions of neurons, or more. In another embodiment, treatment occurs with tissue engineered axonal tracts comprised of tens of axons, thousands of axons, millions of axons, tens of millions of axons, or more. In a further embodiment, multiple direct deliveries of tissue engineered axonal tracts are conducted over time. In one embodiment, tissue engineered axonal tracts are delivered quarterly, bi-annually, annually, or according to various regimens until the nerve injury has been sufficiently treated.

In one aspect, the invention includes a method of treating nerve injury in a subject, comprising contacting a site of nerve injury with a tissue engineered axonal tract wherein when the tissue engineered axonal tract contacts the site of nerve injury, at least one axon from the tissue engineered axonal tract fuses with at least one axon from the subject. In another aspect, the invention includes a method of treating nerve injury in a subject, comprising severing a proximal stump at a site of nerve injury, contacting the proximal stump with a tissue engineered axonal tract, wherein when the tissue engineered axonal tract contacts the proximal stump, at least one axon from the tissue engineered axonal tract fuses with at least one axon from the proximal stump. In yet another aspect, the invention includes a method of treating nerve injury in a subject, comprising contacting a site of nerve injury with a stretch-grown tissue engineered nerve graft (TENG), wherein when the TENG contacts the site of nerve injury, the at least one axon from the TENG fuses with at least one axon from the subject. And, another aspect includes a method of treating nerve injury in a subject comprising contacting a site of nerve injury with a tissue engineered axonal tract and contacting the site of nerve injury with a stretch-grown tissue engineered nerve graft (TENG), wherein when the tissue engineered axonal tract contacts the site of nerve injury, at least one axon from the tissue engineered axonal tract fuses with at least one axon from the subject.

### PEG

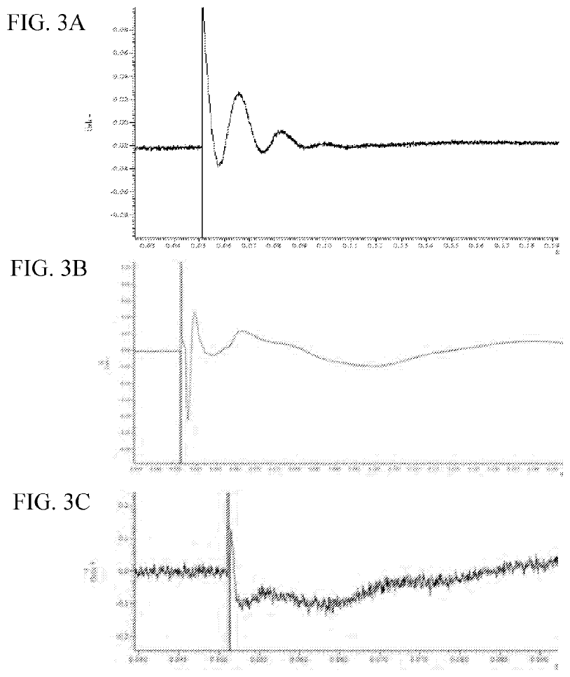

In certain embodiments, polyethylene glycol (PEG) is used to support nerve conduction and fusion of axons from tissue engineered axonal tracts with host axons. PEG-mediated anastomosis of tissue-engineered axons with host axons is illustrated in FIGS. 3A-3C, FIGS. 4A-4B and FIGS. 5A-5F, wherein immediate nerve conduction is demonstrated following fusion of tissue engineered axonal tracts in vivo, ex vivo and in vitro. Effective concentrations and dosages of PEG can be determined by one of ordinary skill in the art. In one non-limiting example, a hypotonic 50% by weight solution of polyethylene glycol (PEG; 3.35 kD) in sterile saline is applied for 2 minutes in PEG-fused animals.

PEG-fusion has been shown to rapidly restore axonal integrity and behavioral function after crush or cut injuries in a rat model, with similar results in a guinea pig model. This PEG-fusion technique has also been demonstrated in several studies to effectively repair neuronal membranes after acute spinal injury. This further corroborates PEG as an effective mediator of axonal repair if given immediately after an acute injury. Indeed, PEG-fusion has also been demonstrated as an effective adjunct to micro-suturing. Behavioral recovery of the sciatic nerve in rats after complete cut was shown to be greatly improved by PEG fusion when compared to a group receiving micro-sutures repair only. PEG-fusion subsequently has been demonstrated to produce rapid behavioral recovery and axonal integrity after ablation and repair by allograft or autograft in rats. PEG has a strong safety record in human medicine, making PEG-fusion technique an excellent potential protocol for human peripheral nerve repair. Despite the promise of PEG-infused nerve repair, the efforts described herein are the first to couple axonal fusion techniques with tissue engineering strategies to enable the “splicing in” of replacement engineered axonal tracts or, eventually, entire replacement engineered nerves. Moreover, using this approach in conjunction with the nerve bridging strategy detailed herein (FIG. 10) is also completely novel.

### Pharmaceutical Compositions

Pharmaceutical compositions of the present invention may comprise the tissue engineered axonal tract as described herein, in combination with one or more pharmaceutically or physiologically acceptable carriers, diluents or excipients. Such compositions may comprise buffers such as neutral buffered saline, phosphate buffered saline and the like; carbohydrates such as glucose, mannose, sucrose or dextrans, mannitol; proteins; polypeptides or amino acids such as glycine; antioxidants; chelating agents such as EDTA or glutathione; adjuvants (e.g., aluminum hydroxide); and preservatives.

Pharmaceutical compositions of the present invention may be administered in a manner appropriate to the disease to be treated (or prevented). The quantity and frequency of administration will be determined by such factors as the condition of the patient, and the type and severity of the patient's disease, although appropriate dosages may be determined by clinical trials.

The tissue engineered axonal tract of the invention to be administered may be autologous, allogeneic or xenogeneic with respect to the subject undergoing therapy.

The tissue engineered axonal tract of the invention can be administered in dosages and routes and at times to be determined in appropriate pre-clinical and clinical experimentation and trials. Compositions may be administered multiple times at dosages within these ranges. Administration of the cells of the invention may be combined with other methods useful to treat the desired disease or condition as determined by those of skill in the art.

The administration of the tissue engineered axonal tract of the invention may be carried out in any convenient manner known to those of skill in the art. The tissue engineered axonal tract of the present invention may be administered to a subject by, injection, implantation or transplantation.

It should be understood that the method and compositions that would be useful in the present invention are not limited to the particular formulations set forth in the examples. The following examples are put forth so as to provide those of ordinary skill in the art with a complete disclosure and description of how to make and use the invention, and therapeutic methods of the invention, and are not intended to limit the scope of what the inventors regard as their invention.

The practice of the present invention employs, unless otherwise indicated, conventional techniques of molecular biology (including recombinant techniques), microbiology, cell biology, biochemistry and immunology, which are well within the purview of the skilled artisan. Such techniques are explained fully in the literature, such as, “Molecular Cloning: A Laboratory Manual”, fourth edition (Sambrook, 2012); “Oligonucleotide Synthesis” (Gait, 1984); “Culture of Animal Cells” (Freshney, 2010); “Methods in Enzymology” “Handbook of Experimental Immunology” (Weir, 1997); “Gene Transfer Vectors for Mammalian Cells” (Miller and Calos, 1987); “Short Protocols in Molecular Biology” (Ausubel, 2002); “Polymerase Chain Reaction: Principles, Applications and Troubleshooting”, (Babar, 2011); “Current Protocols in Immunology” (Coligan, 2002). These techniques are applicable to the production of the polynucleotides and polypeptides of the invention, and, as such, may be considered in making and practicing the invention. Particularly useful techniques for particular embodiments will be discussed in the sections that follow.

### EXPERIMENTAL EXAMPLES

The invention is further described in detail by reference to the following experimental examples. These examples are provided for purposes of illustration only, and are not intended to be limiting unless otherwise specified. Thus, the invention should in no way be construed as being limited to the following examples, but rather, should be construed to encompass any and all variations which become evident as a result of the teaching provided herein.

Without further description, it is believed that one of ordinary skill in the art can, using the preceding description and the following illustrative examples, make and utilize the compounds of the present invention and practice the claimed methods. The following working examples therefore, specifically point out exemplary embodiments of the present invention, and are not to be construed as limiting in any way the remainder of the disclosure.

The materials and methods employed in these experiments are now described.

### In Vivo Methods

Animals (rats) were anesthetized the sciatic nerve exposed. Once the nerve was visualized, compound nerve action potentials (CNAPs) were recorded in isotonic Ca+ free saline by stimulating the entire sciatic nerve in the upper thigh and measuring in the lower thigh. Compound muscle action potentials (CMAPs) were recorded by stimulating the entire sciatic nerve in the upper thigh and recording in the target muscle. The nerve was transected ˜1.5 cm proximal to the knee with micro-dissection scissors. Severed nerve was rinsed in Plasma Lyte A. Hypotonic Ca²⁺-free solution of 1% methylene blue (MB; negative control animals do not receive this solution) in sterile distilled water was applied to coaptation sites for 2-3 minutes at constant flow.

Direct re-anastomosis was performed as a positive control using standard microsurgical techniques. Opposing nerve ends were sutured into place with 9-0 ethylene so that cut ends were opposed without stretching. Fusion of tissue engineered axonal tracts was performed by cutting the sciatic nerve at a second location to excise a 0.5-1.0 cm segment (matching the length of the TENG). The end segments of a TENG (3-D construct embedded in collagen and placed in a nerve guidance tube) were rapidly cut thereby removing the neuronal cell body regions. The TENG ends were treated with Ca²⁺-free MB solution as described herein. Using standard microsurgical techniques, the proximal and distal nerve stumps were gently opposed to the ends of the TENGs. A hypotonic 50% by weight solution of polyethylene glycol (PEG; 3.35 kD) in sterile saline was applied for 2 minutes in PEG-fused animals (negative control animals do not receive PEG treatment). In TENG animals, the nerve guidance conduit was sutured into place with 9-0 ethylene. The nerve was then irrigated with isotonic lactated ringer's solution (contains Ca²⁺). Negative control animals did not receive PEG or MB, but all other steps were identical for the “Direct Re-Anastomosis” and “Fusion of Tissue Engineered Axonal Tracts” groups. In a subset of animals, CMAP and CNAP recordings were immediately taken. In a subset of animals, the fluorescent tract-tracer Flourogold was injected (1.6 μl, 4%, using microinjector) at approximately 0.75 cm distal to the transection site. Muscle and skin were re-opposed and sutured. Animals were returned to the colony, and a subset had nerve conduction assessed at 1 or 8 days post-repair, whereas other animals were left for histological assessment of nerve morphometry at 8 or 14 days post-repair.

### Ex Vivo Methods

Sciatic nerve was exposed as described herein and a 1.0-1.5 cm segment was completely excised and placed in a petri dish in media. Fusion of tissue engineered axonal tracts was performed by rapidly cutting one of the end segments of a TENG (3-D construct embedded in collagen; neurons were transduced to express fluorescent mCherry), thereby removing the neuronal cell body region from one side but leaving the other side intact. The cut ends of the excised nerve and truncated TENG were rinsed in Plasma Lyte A. The cut ends of the excised sciatic and truncated TENF were rinsed with a hypotonic Ca²⁺-free solution of 1% methylene blue (MB; negative control nerves do not receive this solution) in sterile distilled water for 2-3 minutes at constant flow. A hypotonic 50% by weight solution of PEG (3.35 kD) in sterile saline was applied for 2 minutes (negative control nerves do not receive PEG treatment). A surgical sponge containing the fluorescent tracer Lucifer Yellow (LY) was placed in contact with the distal end of the excised sciatic nerve. Nerve-TENG was then irrigated with isotonic lactated ringer's solution (contains Ca2+). Nerve-TENGs were brought to a tissue culture incubator for 1-8 days, after which confocal microscopy was used to assess axon-axon fusion.

The results of the experiments are now described.

### Example 1: Nerve Conduction Following Fusion of Tissue Engineered Axonal Tracts In Vivo

Nerve conduction from the proximal to the distal nerve was measured immediately following PEG-mediated anastomosis of tissue-engineered axons with host axons. Here, a CNAP conducted across the engineered axonal tracts was measured, suggesting fusion of engineered axonal with host axons (FIGS. 3A-3C). Direct re-opposition of proximal and distal stump (positive control; FIG. 3B) with PEG treatment yielded immediate nerve conduction whereas re-opposition (negative control; FIG. 3C) without PEG treatment failed to support nerve conduction. Although the traces clearly reveal recorded signal in (FIG. 3A) and (FIG. 3B), note that the y-axes are different scales in this figure, signifying that the magnitude of the response is currently lower when engineered axonal tracts are “spliced-in” versus direct re-anastomosis.

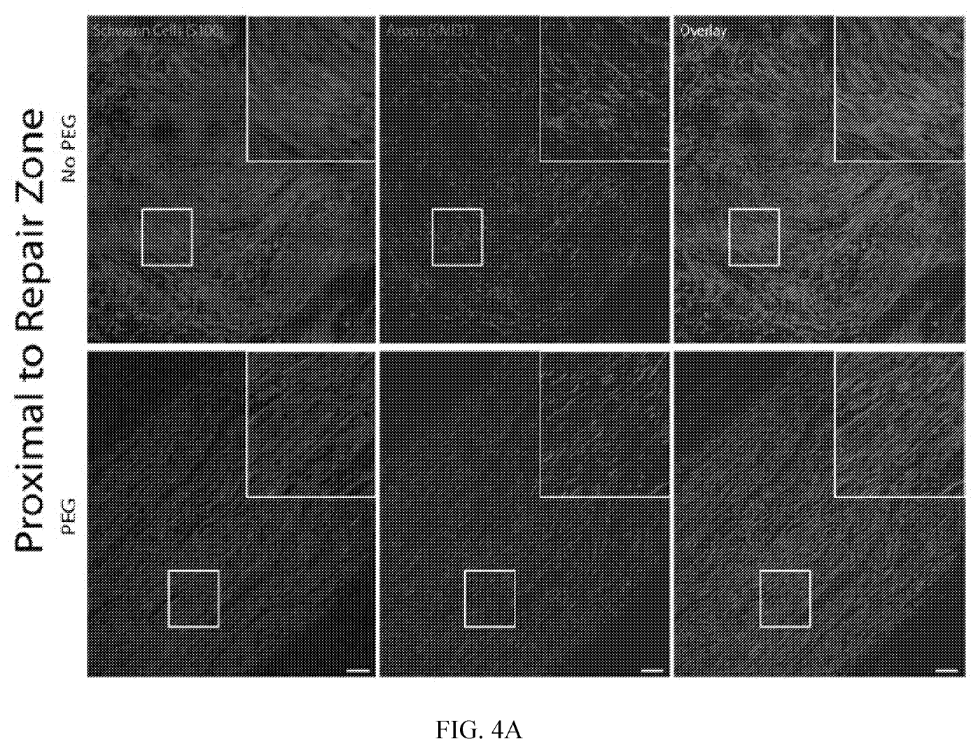

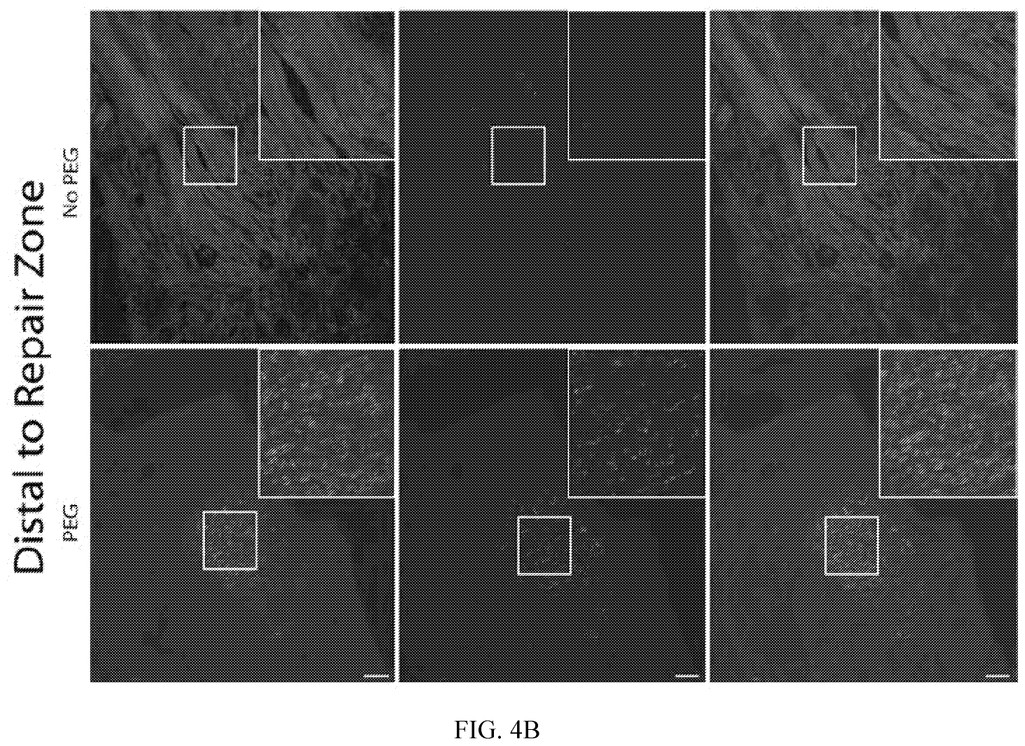

Histological assessment of nerve morphometry was performed in animals that had tissue-engineered axons “spliced-in” using PEG versus no-PEG (negative control). This was performed at 14 days post-fusion, a time point that is more than sufficient for axonal segments distal to the transection site to have degenerated (absent fusion). This revealed that in animals with tissue-engineered axonal tracts with or without PEG, there were dense axons preserved in the nerve segment proximal to the transection site (FIGS. 4A-4B). Moreover, in animals with tissue engineered axonal tracts absent PEG, there was a complete absence of host axons distal to the transection site, indicating complete degeneration. However, in animals receiving tissue-engineered axonal tracts fused using PEG, there were preserved host axons that were clearly visible in the distal segment. This demonstrates that at least a subset of the axons from the tissue engineered axonal tracts fused with transected host axons, thereby preventing their degeneration.

### Example 2: “Axon Fusion” of Engineered Axonal Tracts in Rat Sciatic Nerve Ex Vivo

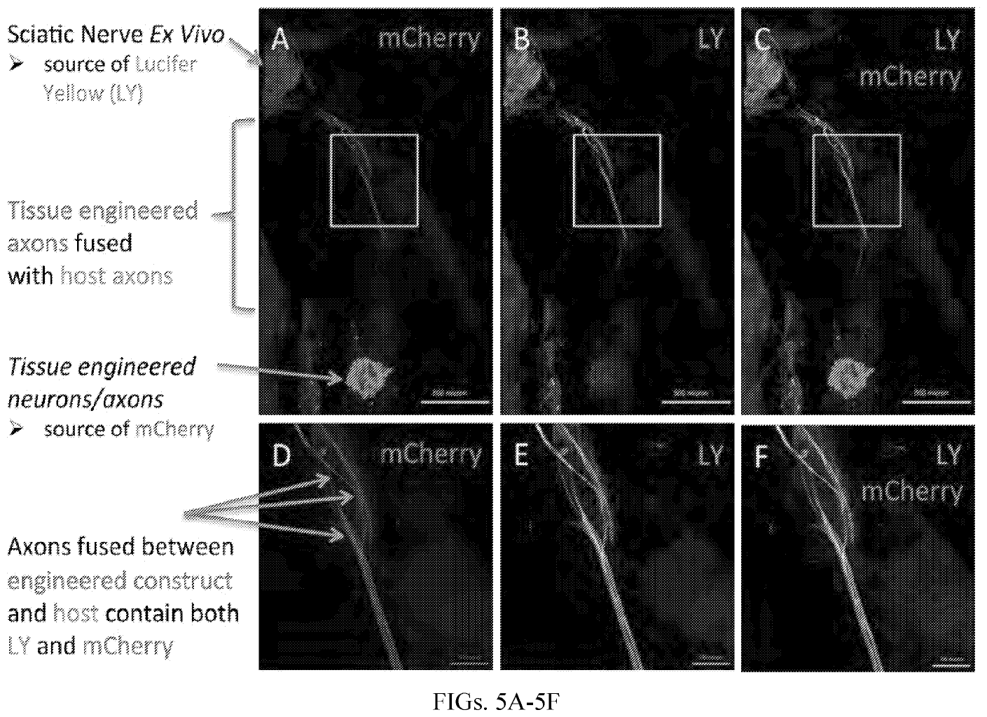

The excised sciatic nerve was opposed to a truncated TENG and treated with PEG as described herein. Here, PEG-mediated fusion resulted in axonal continuity between sciatic axons and TENG axons at 1 day post-fusion (FIGS. 5A-5F). This was demonstrated by central axons containing both LY (transported through the excised host axons) and Cherry (expressed exclusively by the TENG neurons/axons). If “host”/TENG axons had not fused, the ends would have pinched off preventing co-localization of these fluorescent dyes within axons.

### Example 3: “Axon Fusion” of Engineered Axonal Tracts in In Vitro

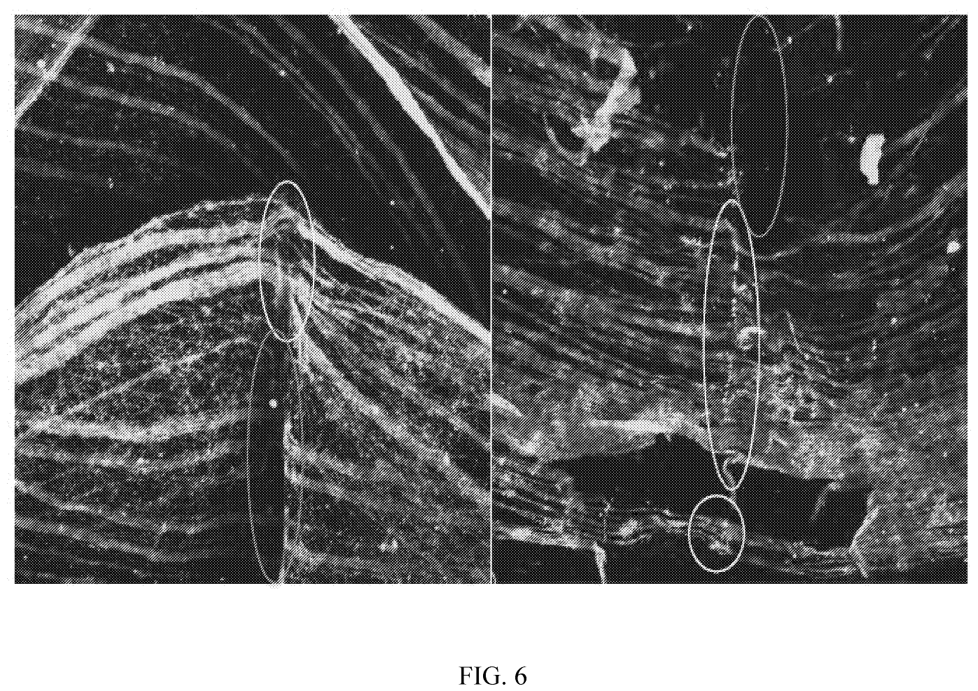

Tissue-engineered axonal tracts were transected and immediately re-fused with PEG in vitro (method described herein). A fluorescent live-dead assay was then used to ascertain axonal continuity across the transection zone, with imaging occurring at 30-60 minutes post-transection/fusion. This revealed that a subset of the axons were continuous, demonstrating immediate re-fusion (FIG. 6).

### Example 4: Applications of Tissue Engineered Axonal Constructs For Neurosurgical Implantation and Axonal Replacement

There has been a long-held belief that axonal disconnection resulted in the inevitable demise (degeneration) of the axonal segment distal to the injury site, as this segment would be physically separated from the neuronal cell body necessary to sustain it. Although it has been known for decades that the axons of many invertebrates are capable of direct “re-anastomosis”, meaning the distal axonal segment can physically re-fuse with the proximal segment, until recently there was no evidence that this was possible for mammalian axons.

In the present study, a methodology is disclosed that allows for the connection or “splicing in” of tissue engineering axonal tracts (FIG. 7), The novel methodology described herein represents a significant advancement beyond other technologies by combining the unique ability to generate long, integrated axonal tracts with adapted techniques for axonal fusion and hence “splicing in” of living, functional tissue engineered axonal tracts into host axonal circuitry. Not only will this technique prevent degeneration of the distal nerve segment and allow virtually immediate recovery of function (FIG. 8), but it creates the possibility of engineering entire nerve segments to be connected directly to the proximal stump to allow for functional recovery even long after the primary injury (FIG. 9). Such “delayed repair” scenarios are extremely common in peripheral nerve surgery, and would necessitate a “fresh cut” of the proximal stump to reveal living host axons capable of being fused with engineered axonal tracts. This new recovery scenario would be in stark contrast to conventional nerve repair marked by degeneration of the distal nerve segment and thus regenerative distances not only across any segmental nerve defect but also along the entire length of the distal nerve segment. At an average axonal regeneration rate of only 1 mm/day, such injuries often necessitate many months or years for recovery, and in cases of ultra-long regenerative distances of tens of centimeters, only very modest—if any—levels of functional recovery are possible due to chronic denervation of the distal nerve segment and muscle target(s).

Therefore, the novel capability described in this disclosure would represent a transformative and disruptive strategy for nerve repair. Even “splicing in” a relatively small number of axons would result in some degree of immediate functional recovery. This will allow some immediate limb control and sensation (versus complete loss of function on the order of months or indefinitely), and equally important, will provide axonal inputs across the distal nerve segments and into the target muscle(s) thereby preventing the detrimental effects of chronic target denervation that ultimately renders muscle unable to re-form neuromuscular junctions even once axons reach the target.

Thus, in the novel approaches disclosed herein, host axons that do not “splice in” with the tissue engineered axonal tracts will be able to regenerate via conventional means, but once they reach appropriate muscles, these targets will he able to receive new axonal inputs—thus this strategy will have a high ceiling for the extent of functional recovery that is ultimately attainable (FIG. 10). Note that these Examples involve peripheral nerve repair; however, analogous repair scenarios may be deployed for repair of the central nervous system (i.e. brain and spinal cord).

### Example 5: “Forced Cell Aggregation” Yields More Robust Axonal Outgrowth, Axonal “Stretch-Growth” and TENG Formation

A novel tissue engineering methodology was devised and validated to more consistently create robust axonal outgrowth, axonal “stretch-growth”, and ultimately TENG formation from neuronal populations. To accomplish this, “forced cell aggregation” was utilized within custom-build pyramidal micro-wells to create “aggregates” or “spheres” of neurons with precise control of the number of neurons—and hence diameter—per aggregate/sphere (FIGS. 12A-12C).

Dissociated neurons were transferred to a chamber containing an array of inverted pyramid micro-wells made in PDMS (Sylguard 184, Dow Corning) cast from a custom-designed 3D printed mold. The wells were then centrifuged at 200 g for 5 min. This centrifugation resulted in forced aggregation of motor neurons with precise control of the number of neurons per aggregate/sphere based on the density of neurons used and the volume added to each well (FIGS. 12A-12C). In one non-limiting example, 12μL of neuronal suspension per well at a density of 300,000-400,000 cells/mL was suitable to create aggregate/spheres of appropriate diameter for seeding, neuronal survival, axonal extension, and axonal “stretch-growth”.

The aggregates/spheres were carefully placed at the towing membrane—base membrane border in custom mechanobioreactors and allowed to adhere at 37° C., 5% CO₂, before the chambers were flooded with media. The media for motor neuron culture and axon stretch-growth was Neurobasal media with 10% FBS+hydrocortisone, growth factor cocktail (BDNF, CNTF, Cardiotrophin 1, GDNF), B-27, L-glutamine, Sodium Pyruvate, forskolin, mitotic inhibitors, isobutylmethylxanthine, and beta mercaptoethanol; this media was “glial conditioned” prior to use in motor neuron cultures. For motor neurons in co-culture with DRG neurons, the above media was further supplemented with NGF and glucose. Seeded mechanobioreactors were allowed to grow over at least several days in vitro prior to engaging the micro-stepper motor for axonal-stretch growth.

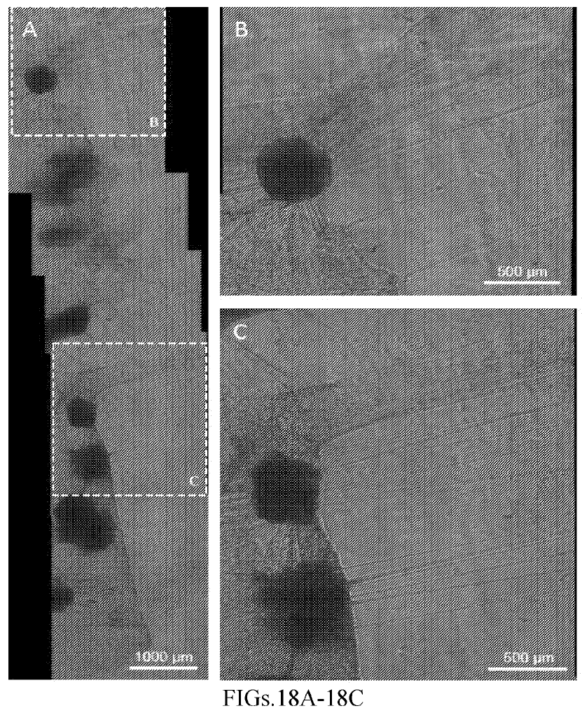

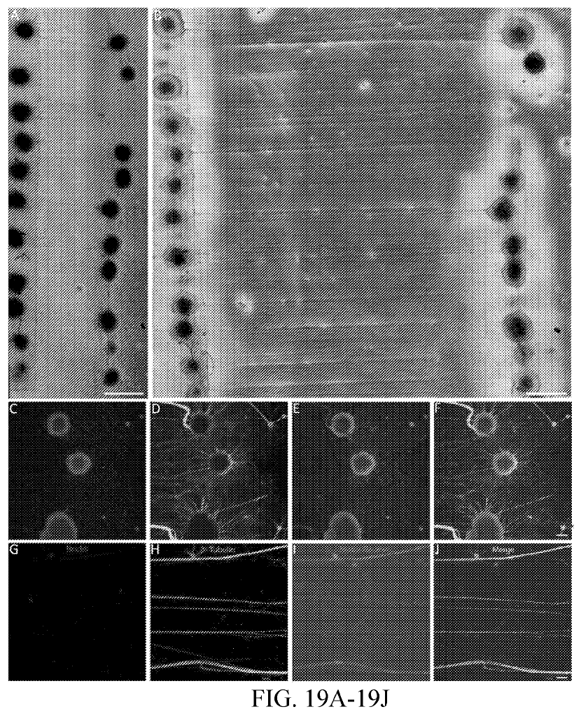

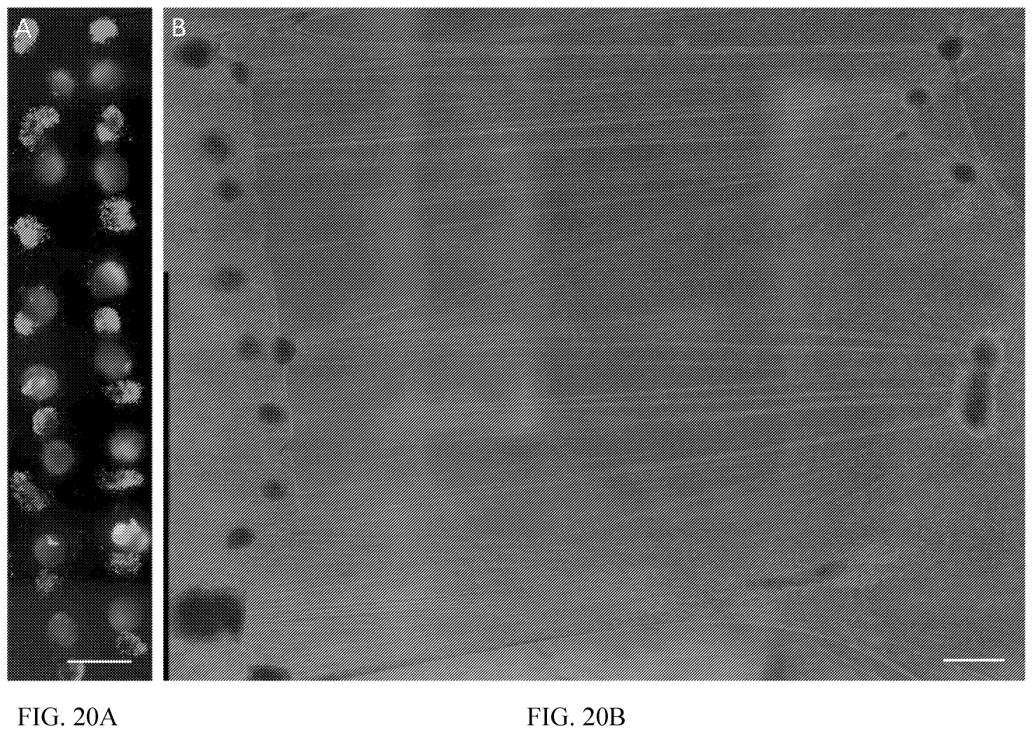

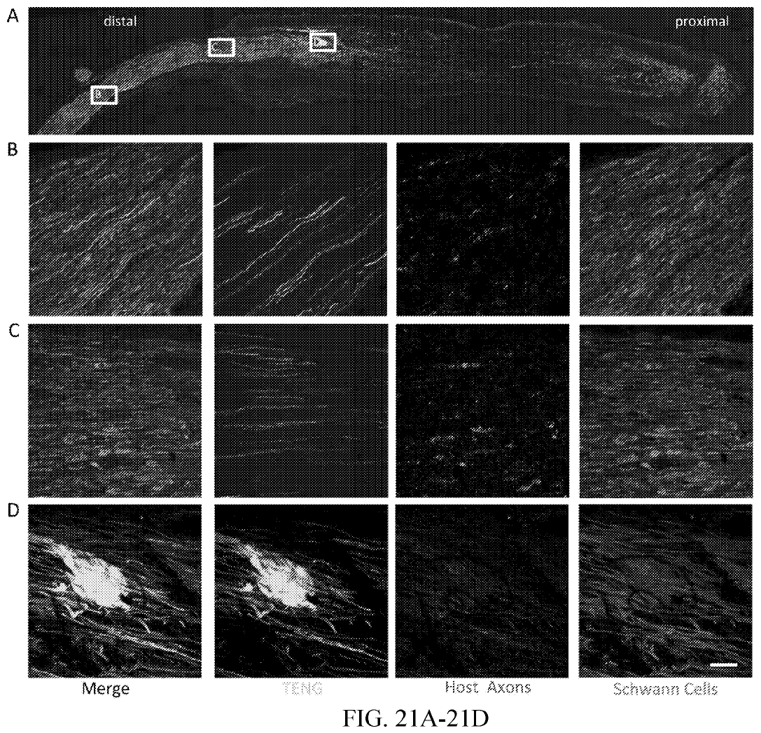

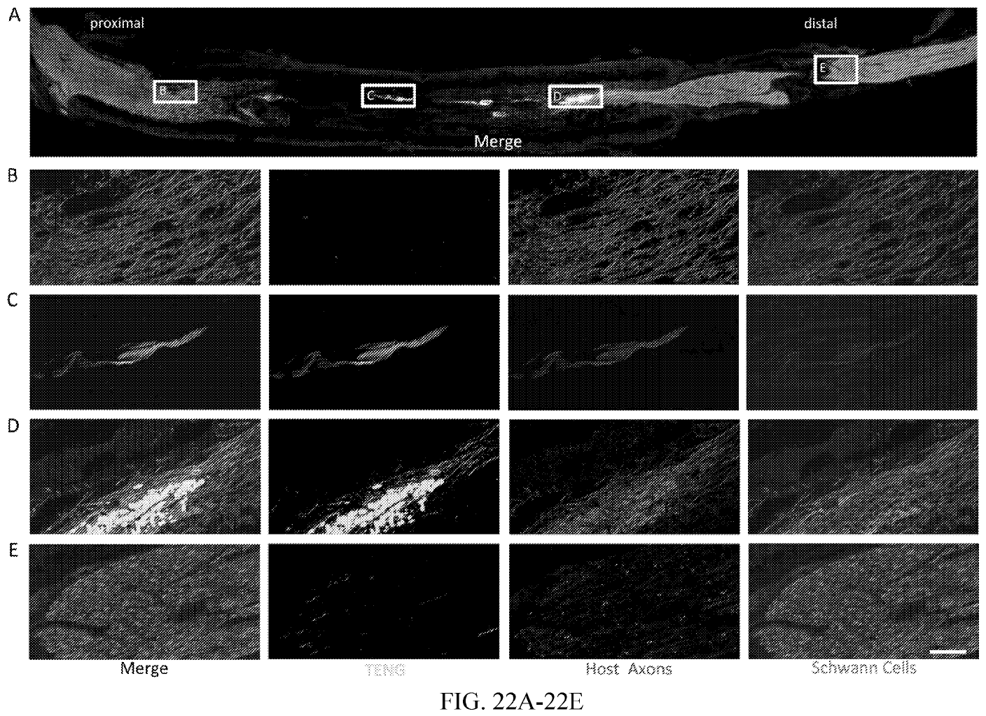

This neuronal “forced aggregate” methodology resulted in the formation of robust axonal extension as compared to dissociated neurons (FIGS. 13-15). Moreover, axons projecting from these “forced aggregates” exhibited robust axonal “stretch-growth”, on par with that generated from axons projecting from sensory dorsal root ganglia neurons (FIGS. 18A-18C).

This improved axonal outgrowth was further verified by immunocytochemistry and confocal microscopy to label these aggregate motor neurons (versus dissociated motor neurons) using antibodies recognizing all axons (beta-tubulin III; green) and all cell nuclei (Hoechst; blue) (FIG. 15). This revealed a significant increase in the density and extent of axonal outgrowth when motor neuron aggregates were in co-culture with sensory neuron ganglia (versus dissociated motor neurons in co-culture with sensory neuron ganglia).

### Example 6: Tissue Engineered Axon-Based “Living Scaffolds” Preserve Regenerative Pathway and Capacity for Muscle Reinnervation Following Peripheral Nerve Injury

Combat-related trauma to the extremities often results in extensive damage to the warfighter's limbs, with traumatic peripheral nerve injury (PNI) among the most predominant injuries sustained. With current strategies for surgical repair of PNI, recovery of motor and sensory function is generally inadequate owing to a lack of treatments aimed at preserving the regenerative environment of distal nerve segments and the reinnervation capacity of target muscle(s). Indeed, peripheral nerve regeneration is a race against time as long distances for axonal re-growth (i.e. from a point of transection to end targets) often requires many months, over which time endogenous Schwann cells (SCs) and myofibers gradually loose their capacity to support axonal regeneration and reinnervation, respectively. Also, inadequate bridging strategies across segmental nerve defects can result in diminished axonal regenerative capacity, creating a low ceiling for the extent of possible functional recovery.

To address this need, tissue engineered nerve grafts (TENGs) consisting of living neurons and aligned axonal tracts were developed which are routinely generated in custom-built mechanobioreactors at densities of >100,000 axons and lengths of >5 cm within 2 weeks through the controlled process of axon “stretch-growth”. TENG axons mimic the developmental action of “pioneer” axons, where targeted axonal outgrowth can be achieved along pre-existing axonal tracts in vivo. In the current study, allogeneic TENGs were created at lengths ranging from 1-5 cm and used to bridge segmental nerve defects in rats or pigs, with terminal time points ranging from 2 weeks to 9 months. Histological outcomes included axon re-growth, SC presence and alignment, and dorsal root ganglia (DRG) and spinal motor neuron (SMN) health. Functional outcomes such as nerve conduction and muscle action potentials were assessed at chronic time points. As previously reported, TENGs exploited the newfound mechanism of axon-facilitated axon regeneration to serve as living bridges across 5 cm segmental nerve defects to facilitate muscle reinnervation in porcine models of major PNI. The focus of the current study was to evaluate the effects of TENGs on preserving host SC regenerative capacity, DRG/SMN health, and myofibers mass in rat and pig models of PNI. First, it was found that axons projecting from TENGs grew along host SCs in otherwise denervated nerve segments—a mechanism not possible with autograft repairs—that served to maintain pro-regenerative SC alignment over at least several months. Next, it was found that acute improvements in SMN health in animals repaired with TENGs or autografts versus those repaired with acellular nerve guidance tubes (NGTs) or target muscle mass and fiber size distributions in animals repaired with TENGs or autografts versus NGTs alone.

The data provided here establish that TENGs uniquely act as “living scaffolds” to promote a favorable environment for nerve regeneration by maintaining pro-regenerative capacity of SCs, health of SMNs, and muscle fiber mass, collectively raising the ceiling for potential levels of functional recovery beyond that attainable by conventional surgical repair strategies.

The recitation of a listing of elements in any definition of a variable herein includes definitions of that variable as any single element or combination (or subcombination) of listed elements. The recitation of an embodiment herein includes that embodiment as any single embodiment or in combination with any other embodiments or portions thereof.

The disclosures of each and every patent, patent application, and publication cited herein are hereby incorporated herein by reference in their entirety. While this invention has been disclosed with reference to specific embodiments, it is apparent that other embodiments and variations of this invention may be devised by others skilled in the art without departing from the true spirit and scope of the invention. The appended claims are intended to be construed to include all such embodiments and equivalent variations.

1. A method of treating nerve injury in a subject, the method comprising:
contacting a site of nerve injury with a tissue engineered axonal tract,
wherein when the tissue engineered axonal tract contacts the site of nerve injury, at least one axon from the tissue engineered axonal tract fuses with at least one axon from the subject.

2. A method of treating nerve injury in a subject, the method comprising:
severing a proximal stump at a site of nerve injury,
contacting the proximal stump with a tissue engineered axonal tract, wherein when the tissue engineered axonal tract contacts the proximal stump, at least one axon from the tissue engineered axonal tract fuses with at least one axon from the proximal stump.

3. A method of treating nerve injury in a subject, the method comprising:
contacting a site of nerve injury with a stretch-grown tissue engineered nerve graft (TENG), wherein when the TENG contacts the site of nerve injury, the at least one axon from the TENG fuses with at least one axon from the subject.

4. A method of treating nerve injury in a subject, the method comprising:
contacting a site of nerve injury with a tissue engineered axonal tract and a stretch-grown tissue engineered nerve graft (TENG),
wherein when the tissue engineered axonal tract contacts the site of nerve injury, at least one axon from the tissue engineered axonal tract fuses with at least one axon from the subject.

5. The method of claim 1, further comprising regeneration of a distal nerve segment in the subject.

6. The method of claim 1, wherein the nerve injury is selected from the group consisting of: peripheral nerve injury, brain injury, and spinal cord injury.

7. The method of claim 1, further comprising the use of polyethylene glycol (PEG) to support nerve conduction and fusion of axons.

8. The method of claim 7, further comprising the application of a hypotonic 50% by weight solution of PEG.

9. The method of claim 1, wherein the nerve injury is a result of a neurodegenerative disease.

10. The method according to claim 1, wherein the TENG is a forced aggregation TENG.

11. A composition comprising a tissue engineered axonal tract.

12. The composition of claim 11, wherein the tissue engineered axonal tract is generated from a forced aggregation TENG.

13. A method of generating a tissue engineered axonal tract, the method comprising
severing at least one neuronal cell body from a stretch-grown tissue engineered nerve graft (TENG), wherein when the at least one neuronal cell body is severed, a tissue engineered axonal tract is generated.

14. The method of claim 13, wherein the stretch-grown tissue engineered nerve graft (TENG) is a forced cell aggregation TENG.

15. A tissue engineered axonal tract made by the method according to claim 13.
